(19) United States
(12) Patent Application Publication
(10) Pub. No.: US 2015/0099001 A1
(43) Pub. Date: Apr. 9, 2015

(54) Nanocell Drug Delivery System

(71) Applicant: Massachusetts Institute of Technology, Cambridge, MA (US)

(72) Inventors: Shiladitya Sengupta, Waltham, MA (US); Ganlin Zhao, Arlington, MA (US); Ishan Capila, Ashland, MA (US); David Eavarone, North Quincy, MA (US); Ram Sasisekharan, Cambridge, MA (US)

(21) Appl. No.: 14/480,123

(22) Filed: Sep. 8, 2014

Related U.S. Application Data
(63) Continuation of application No. 13/745,259, filed on Jan. 18, 2013, now abandoned.
Application No. 13/745,259 is a continuation of application No. 12/794,468, filed on Jun. 4, 2010, now abandoned.
Application No. 12/794,468 is a continuation of application No. 11/495,947, filed on Jul. 28, 2006, now abandoned.
Application No. 11/495,947 is a continuation-in-part of application No. 11/070,731, filed on Mar. 2, 2005, now abandoned.
(60) Provisional application No. 60/549,280, filed on Mar. 2, 2004.

(51) Int. Cl. A61K 9/51; A61K 31/09; A61K 47/48; A61K 31/704

(52) U.S. Cl. CPC A61K 9/5153; A61K 31/704; A61K 9/51; A61K 31/09; A61K 47/482; A61K 9/5123
USPC 424/490; 514/34; 428/402

(57) ABSTRACT

Nanocells allow the sequential delivery of two different therapeutic agents with different modes of action or different pharmacokinetics. A nanocell is formed by encapsulating a nanocore with a first agent inside a lipid vesicle containing a second agent. The agent in the outer lipid compartment is released first and may exert its effect before the agent in the nanocore is released. The nanocell delivery system may be formulated in pharmaceutical composition for delivery to patients suffering from diseases such as cancer, inflammatory diseases such as asthma, autoimmune diseases such as rheumatoid arthritis, infectious diseases, and neurological diseases such as epilepsy. In treating cancer, a traditional antineoplastic agent is contained in the outer lipid vesicle of the nanocell, and an antiangiogenic agent is loaded into the nanocore. This arrangement allows the antineoplastic agent to be released first and delivered to the tumor before the tumor's blood supply is cut off by the antianiogenic agent.

### CROSS-REFERENCE TO RELATED APPLICATIONS

The present application is a continuation application of U.S. patent application Ser. No. 13/745,259, filed Jan. 18, 2013, which is a continuation application of U.S. patent application Ser. No. 12/794,468, filed Jun. 4, 2010, which is a continuation application of U.S. patent application Ser. No. 11/495,947, filed Jul. 28, 2006, which is a continuation-in-part of U.S. patent application Ser. No. 11/070,731, filed Mar. 2, 2005, which claims priority to U.S. Provisional Application No. 60/549,280, filed on Mar. 2, 2004. The disclosures of each of the prior applications are incorporated by reference herein.

### GOVERNMENT SUPPORT

This invention was made with government support awarded by the National Institutes of Health under NIG Grant No. 5-R01-CA090940-03. The U.S. Government has certain rights in the invention.

### BACKGROUND OF THE INVENTION

The prerequisites for rational drug therapy are an accurate diagnosis, knowledge of the pathophysiology of the disease, the knowledge of basic pharmacotherapeutics in normal and diseased people, and the reasonable expectations of these relationships so that the drug's effects can be anticipated (DiPiro et al. Eds. Pharmacotherapy—A pathophysiologic approach, 2nd Ed). Advances made in biomedical sciences, in terms of the genome, proteome, or the glycome, have unraveled the molecular mechanisms underlying many diseases, and have implicated a complex network of signaling cascades, the transcriptome, and the glycome that are distinctly altered. In most pathophysiological conditions, this may manifest as valid targets for modulation for a recovery or the loss of function, resulting in a therapeutic outcome. However, the complexity lies in the involvement of distinct pathways at the diseased tissue, or even spatially distinct target cells in the diseased tissue, or temporal events occurring within a diseased tissue that manifests in the final phenotype. The logical strategy is to target the disease at multiple levels, which can be achieved using combination therapies of multiple active agents or drugs. However, this is often not an optimal strategy in most conditions, being limited by patient compliance in taking too many drugs, or by drug-drug interactions at the level of pharmacokinetics (absorption, distribution, biotransformation, and excretion) and pharmacodynamics (biochemical and physiological effects of drugs and their mechanisms of action), or toxicology (Goodman and Gilman's The Pharmacological Basis of Therapeutics, 9th Edition). Such interactions can reduce the actual therapeutic effect of an active agent or increase its toxicity, the ratio of which is defined as the therapeutic index. An inventive solution to the above limitations would certainly revolutionize medicine and therapeutics.

To better understand the limitation of modern medicine, an appropriate example is the treatment of tumors. One-third of all individuals in the United States will develop cancer. Although the five-year survival rate has risen dramatically to nearly fifty percent as a result of progress in early diagnosis and therapies, cancer still remains second only to cardiac disease as a cause of death in the United States. Twenty percent of Americans die from cancer—half due to lung, breast, and colon-rectal cancer.

Designing effective treatments for patients with cancer has represented a major challenge. The current regimen of surgical resection, external beam radiation therapy, and/or systemic chemotherapy has been partially successful in some kinds of malignancies, but has not produced satisfactory results in others. In some malignancies, such as brain malignancies, this regimen produces a median survival of less than one year. For example, 90% of resected malignant gliomas recur within two centimeters of the original tumor site within one year.

Though effective in some kinds of cancers, the use of systemic chemotherapy has had to minor successes in the treatment of cancers of the colon-rectum, esophagus, liver, pancreas, and kidney, and skin. A major problem with systemic chemotherapy for the treatment of these types of cancers is that the systemic doses required to achieve control over tumor growth frequently result in unacceptable systemic toxicity. Efforts to improve delivery of chemotherapeutic agents to the tumor site have resulted in advances in organ-directed chemotherapy, for example, by continuous systemic infusion. However, continuous infusions of anticancer drugs generally have not shown a clear benefit over pulse or short-term infusions. The anti-neoplastic or chemotherapeutic agents currently used in the clinic include (a) alkylating agents, such as mechlorethamine, cyclophosphamide, ifosfamide, melphaan, chlorambucil, hexamethylmelamine, thiotepa, busulfan, carmustine, lomustine, semustine, streptozocin, dacarbazine, etc.; (b) antimetabolites, such as methotrexate, 5-FU, FudR, cytarabine, 6 MP, thioguanine, pentostatin, etc.; (c) natural products, such as taxol, vinblastine, vincristine, etoposide, teniposide, etc.; (d) antibiotics such as dactinomycin, daunorubicin, doxorubicin, bleomycin, plicamycin, mitomycin c, etc.; (e) enzymes such as L-asparaginase, heparinases, chondroitinases, etc.; (f) interferons and interleukins, such as interferon-α, interferon-γ, tumor necrosis factor, etc.; (g) platinum coordination complexes such as cisplatin, carboplatin or their derivatives; and (h) other miscellaneous agents such as mitoxantrone, bischloroethyl nitrosourea, hydroxyurea, chloroethyl-cyclohexyl nitrosourea, prednisone, diethylstilbestrol, medroxyprogesterone, tamoxifen, mitotane, procarbazine, aminoglutethimide, progestins, androgens, antiadrogens, Leuprolide, etc.

A recent advancement in anti-tumor therapy has been the identification of angiogenesis as a key step in the development of a tumor. Angiogenesis, the development of new blood vessels from an existing vascular bed, underlies the rapid expansion of a tumor and the development of distant metastasis (Folkman, Nat Med, January 1995; 1:27-31). When tumor reaches a stage of 1-2 mm³ in volume, it needs nutrients for further growth. The cells at the core of the tumor start dying leading to a necrotic core that is rich in growth factors and pro-angiogenic signals that lead to the recruitment of endothelial cells from the nearest blood vessel. Executed in distinct sequential steps, angiogenesis is the culmination of spatio-temporal interactions between the tumor cells, the extra-cellular matrix, and the endothelial cells, brought about by the interplay of multiple mediators (Griffoen and Molema, Pharmacol. Review, June 2000; 52:237-68). The understanding of the events underlying this complex process and the elucidation of the mechanisms of action of some of the mediators has opened up the exciting possibility of therapeutic targeting of angiogenesis as a novel strategy for tumor management, with over sixty compounds in clinical stages of development.

Currently there are two classes of angiogenesis inhibitors—direct and indirect. Direct angiogenesis inhibitors, such as vitaxin, angiostatin, endostatin, combretastatin, 2-methoxyestradiol, avastin, canstatin, and others, prevent endothelial cells from proliferating, migrating, or forming tubes, or allow the cell to avoid cell death in response to the tumor-secreted angiogenic factors. Indirect angiogenesis inhibitors generally prevent the expression of or block the activity of a tumor protein that activates angiogenesis, or block the expression of its receptors on endothelial cells (Kerbel and Folkman, Nature Reviews Cancer, October 2002; 727-739). The end result of an anti-angiogenic therapy in both cases is the shutdown of vascular supply to the growing tumor resulting in starving the tumor. Therefore, antiangiogenic therapy results in hypoxia in the tumor (Yu J L et al, Science, February 2002; Vol 295:1526-1528). To overcome this hypoxic situation, tumors starts producing growth factors, which also exert an angiogenic effect similar to the angiogenic effect when the tumor was much smaller. In the clinic, this translates into a spurt in the growth of the tumor as soon as the anti-angiogenic therapy is stopped (Boehm et al., Nature 390:404-407, November 1997). The same growth factors can also prevent some of the tumor cells from undergoing apoptosis or cell death. Furthermore, tumor hypoxia, due to abnormal or sluggish blood flow within areas of the solid tumors, can result in both microenvironment-mediated radiation and chemotherapeutic drug resistance (Yu et al., Differentiation, December 2002: Vol 70:599-609). It is also possible that variant tumor cells that are less vessel dependent and may therefore be selected for over time by successful antiangiogenic therapy. This would result in the loss of response or attenuated response to more traditional forms of chemotherapy. This can be overcome by the combined use of bioreductive hypoxic cell cytotoxic drugs and antiangiogenics (Yu J L, Differentiation December 2002; 70:599-609). The use of a combination therapy of antineoplastic or chemotherapeutic agents with antiangiogenics for the treatment of cancer/tumor is disclosed in multiple patents applications (See, e.g., U.S. Pat. Nos. 6,147,060; 6,140,346; and 5,856,315; 5,731,325; 5,710,134, and 5,574,026; each of which is incorporated herein by reference; U.S. Patent Applications 20020041880; 20020107191; 20020128228; 20020111362; each of which is incorporated herein by reference). However, there remains a need for a drug delivery system for delivering combination therapies so that each agent provides the desired maximal effect. Such a system would be useful not only in the treatment of cancer but would also find use in the treatment of other diseases such as autoimmune disease (e.g., rheumatoid arthritis), inflammatory diseases (e.g., asthma), neurological diseases (e.g., epilepsy), and ophthamological diseases (e.g., diabetic retinopathy).

### SUMMARY OF THE INVENTION

The present invention stems from the recognition that many drugs used in combination therapies act via different mechanisms and/or on different time scales. Therefore, if a drug in a combination therapy cannot reach its target or does not reach its target at the appropriate time, much, if not all, of the efficacy of the drug is lost. For example, in treating cancer with a combination of a more traditional anti-neoplastic agent and an anti-angiogenic agent, the anti-neoplastic agent should optimally get to the tumor to exert its effect before the anti-angiogenic agent prevents blood flow, which carries the anti-neoplatic agent, from reaching the tumor cells. If the anti-neoplatic agent does not reach the tumor before the functional vasculature is shut down by the anti-angiogenic agent, the patient will suffer from the side effects of the anti-neoplastic agent without receiving any of its benefits. Therefore, in cancer as well as many other diseases, there is a need for a drug delivery system that will allow for the delivery of multiple agents at different time intervals.

The present invention provides for a drug delivery system in which one agent can be delivered before or after another agent in a combination therapy. The drug delivery system is based on the concept of a balloon within a balloon. A nanocore (e.g., a nanoparticle, nanotube, nanowire, quantum dot, etc.) containing a pharmaceutical agent is encapsulated in a lipid vesicle, matrix, or shell that contains another pharmaceutical agent, to form a nanocell. The pharmaceutical agent in the outer portion of the nanocell (e.g., lipid vesicle, shell, or matrix) is released first followed by the release of the second pharmaceutical agent with the dissolution and/or degradation of the nanocore. The inventive nanocells range in size from 10 nm to 500 micrometers in their largest diameter, preferably from 80 nm to 50 micrometers in their largest diameter.

For example, in treating cancer, an antiangiogenic agent is loaded inside the lipid vesicle and is released before the anti-neoplastic/chemotherapeutic agent inside the inner nanoparticle. This results in the collapse of the vasculature feeding the tumor, and also leads to the entrapment of the anti-neoplastic agent-loaded nanocores inside the tumor with no escape route. The anti-neoplastic agent is released slowly resulting in the killing of the nutrient-starved tumor cells. In other words, this double balloon drug delivery system allows one to load up the tumor with an anti-neoplastic agent and then cut off the blood supply to the tumor. This sequential process results in the entrapment of the toxic chemotherapeutic/antineoplastic agent within the tumor, leading to increased and selective toxicity against the tumor cells, and less drug is present in the systemic circulation, since it cannot leak out from the functionally avascular tumor site, resulting in less side effects. This technique also overcomes the hypoxia caveat, as the tumor-entrapped cytotoxic chemotherapeutic cell kills off the tumor cells that would have otherwise survived in the hypoxic growth factor-rich environment resulting from the vascular shutdown.

The inner nanoparticle (also known as the nanocore) is approximately 10-20000 nm in its greatest dimension and contains a first therapeutic agent encapsulated in a polymeric matrix. These nanocores are prepared using any of the materials such as lipids, proteins, carbohydrates, simple conjugates, and polymers (e.g. PLGA, polyesters, polyamides, polycarbonates, poly(beta-amino esters), polyureas, polycarbamates, proteins, etc.) and methods (e.g., double emulsion, spray drying, phase inversion, etc.) known in the art. Pharmaceutical or diagnostic agents can be loaded in the nanocore, or covalently linked, or bound through electrostatic charges, or electrovalently conjugated, or conjugated through a linker. The result is a slow, sustained, and/or delayed release of the agent(s) from the nanocore. Preferably, if the agent is covalently linked to the nanocore, the linker or bond is biodegradable or hydrolysable under physiological conditions, e.g., susceptible to enzymatic breakdown. The nanocore can be a substantially spherical nanoparticle, nanoliposome, a nanowire, a quantum dot, or a nanotube.

To form a nanocell, the nanocores are coated with a lipid with a second therapeutic agent partitioned in the lipid phase. Nanocells may also be formed by coating the nanocores with a distinct polymer composition with a second therapeutic agent. Preferably, the nanoshell or the surrounding matrix of the nanocell should comprise a composition that allows a fast release of the agent/s that it entraps. Therefore, in certain embodiments, the effect of this agent begins before the active agent loaded in the nanocore reaches therapeutic level. Therefore, the second therapeutic agent is outside the nancore but inside the lipid membrane of the nanocell, which is approximately 50-20000 nm in its greatest diameter. The nanocell may be further coated to stabilize the particle or to add targeting agents onto the outside of the particle.

To further illustrate aspects of the present invention, in certain embodiments of the subject nanocells, there is provided a drug delivery particle for the temporally controlled delivery of two different therapeutic agents, comprising (i) a nanocore including a first therapeutic agent; and (ii) an outer layer coating said nanocore, said outer layer including a second therapeutic agent. In such embodiments, the second therapeutic agent is released first, followed by release of the first therapeutic agent from the nanocore.

In certain preferred embodiments, the nanocore is a nanoparticle comprising a polymeric matrix containing the first therapeutic agent, and the first therapeutic agent is released upon the dissolution or degradation of said polymeric matrix. The outer layer allows a fast release of the second therapeutic agent, such that the second therapeutic agent is released first, followed by a slower release of the first therapeutic agent from the nanoparticle. In this embodiment, the pharmacological effect of the second therapeutic agent begins before the first therapeutic agent reaches therapeutic levels in the patient. For instance, the second therapeutic agent can be released from the drug delivery particle on a time scale of minutes, while the release of the first therapeutic agent from the core can be on a substantially longer time scale.

In certain preferred embodiments, the outer layer includes or is formed by a lipid vesicle including the second therapeutic agent.

In certain embodiments, the nanocells can be used as part of the treatment of an acute condition, such as in asthma, where 25% of maximal loading of the second therapeutic agent is released before 10% of maximal loading of First therapeutic agent is released, e.g., as measured by in vitro or in vivo studies, and even more preferably, at least 40%, 50%, 60% or even 75% (in some embodiments) of maximal loading of second therapeutic agent is released before 10% or less of maximal loading of first therapeutic agent is released. In certain embodiments, at least 25%, 40%, 50%, 60% or even 75% of maximal loading of the second therapeutic agent is released before 2% or less of maximal loading of the first therapeutic agent is released.

In certain embodiments, such as for the treatment of acute conditions, the components of the nanocell are selected such that the rate of release of second therapeutic agent is at least about twice as fast as the rate of release of first therapeutic agent. Even more preferably, the rate of release of second therapeutic agent can be at least about 3, 5, 10 or even 50 times faster than first therapeutic agent.

For certain embodiments in which the subject nanocells are used for the treatment of chronic disease with more than one compartments (such as in cancer, which has a tumor and a stroma, or rheumatoid arthritis and psoriasis, which has proliferative cells and angiogenesis), the components of the nanocell can be selected such that about 25% of maximal loading of the second therapeutic agent is released before 10% of maximal loading of first therapeutic agent is released, and more preferably at least 40%, 50%, 60% or even 75% of maximal loading of the second therapeutic agent is released before 10% or less of maximal loading of the first therapeutic agent is released. In certain embodiments, at least 25%, 40%, 50%, 60% or even 75% of maximal loading of the second therapeutic agent is released before 2% or less of maximal loading of the first therapeutic agent is released.

In certain embodiments where the nanocells are used in the treatment of a complex chronic disease with more than one compartments (such as in cancer, rheumatoid arthritis and psoriasis), the components of the nanocell can be selected such that the rate of release of second therapeutic agent is at least abouttwice as fast at the rate of release of first therapeutic agent. Even more preferably, the rate of release of second therapeutic agent can be 3, 5, 10 or even 50 times faster than first therapeutic agent.

Any two or more pharmaceutical agents may be delivered using the inventive nanocells. Preferably, one agent or combination of agents is optimally delivered before a second agent or combination of agents. In certain embodiments, the agents may differ in mode of action or target. For example, in certain embodiments, the agent in the nanocore may inhibit a signaling pathway, and the agent in the outer compartment of the nanocell effects a different pathway or a different signal in the same pathway. The two agents may act synergistically. In other embodiments, the agents may differ in their pharmacokinetics. For example, in the treatment of arthritis, methotrexate or colchicine is encapsulated in a nanocore, and an anti-angiogenic agent is in the outside lipid portion of the nanocell. In treating asthma or chronic obstructive pulmonary disease (COPD), an anti-inflammatory agent (e.g., corticosteroid, lipooxygenase inhibitor, mast cell stabilizer) is provided in the nanocore, and a bronchodilator (e.g., a β-agonist) is provided in the outer compartment of the nanocell. In delivering agents to the brain, in order to cross the blood-brain barrier, a chaotropic agent or other agent that allows drugs to cross the blood brain barrier is provided in the outside portion of the particle, and a neuroactive agent such as an anti-seizure agent is provided in the nanocore. In other embodiments, the nanocells may be used to treat a patient with cystic fibrosis. For example, the nanocell may be used to deliver an antibiotic and an anti-inflammatory agent. In other embodiments, the nanocells are used as vehicles for delivering vaccines, for example, an antigen may be loaded in the nanocore, and an inflammatory agent such as an adjuvant may be included in the outer portion of the nanocell.

In certain embodiments, the drug delivered from the core of the nanocell, e.g., the so-called “first therapeutic agent” above, is not very water soluble. For instance, the drug may have a solubility of less than 0.1 mg/ml in water at 25° C., and can be less than 10 μg/ml or even 1.0 μg/ml in water at 25° C. For those embodiments in which the drug is generally water soluble, a less water soluble prodrug version can be used in the nanocell, which is converted to the drug either prior to or after release from the core. For instance, the prodrug can have a log P value at least 0.5 log P units more than the log P value for the parent drug, and even more preferably at least 1 log P unit greater. In still other embodiments, water soluble drugs can be delivered from the core by forming the core with a version of the drug that has been covalently or non-covalently linked to the polymer of the core as a means for reducing the water solubility of the drug.

In another aspect, the present invention provides pharmaceutical composition with the inventive nanocells. These compositions may also include other pharmaceutically acceptable excipients. The compositions may be in the form of tablets, suspensions, solutions, capsules, emulsions, etc.

Nanocells for treatment of above conditions may be administered, for example, as injectables (iv, ip, icy, ia) or as respirables. The size of nanocells for injections desirably ranges between about 50 nm and 500 nm, while that for respirables will typically range from about 2-50 microns. Each respirable nanocell may have 1-500 nanocores, and preferably will have between 1-200 nanocores.

The present invention also provides methods of treating various diseases by administering nanocells loaded with the appropriate pharmaceutical agents to a patient suffering from a disease. These methods includes methods of treating cancer, inflammatory diseases, ophthalmological diseases, neurological disease, infectious diseases, and autoimmune diseases. The nanocells are loaded with the amount of agent needed to deliver a therapeutically effective amount of the agent and achieve a desired result. As would be appreciated by one of skill in this art, the agents and dosages used as well as the excipients in the nanocells will be depend on the patient being treated (including kidney and liver functions), the disease being treated, the various pharmacological and pharmacokinetic characteristics of the agents to be delivered, clinical setting, mode of administration, etc. The nanocells may be administered using any routes of administration known. In certain embodiment, the nanocells are delivered parenterally. In other embodiments, the nanocells are delivered inhalationally, for example, using an atomizer, spinhaler, or diskhaler.

### (i) Asthma

In some embodiments, nanocells for use in the treatment of asthma have acore constructed of biodegradable polymers that releases a corticosteroid in a time scale of hours and days. Desirably, the matrix surrounding the nanocore may be constructed out of a water-soluble compound, polymers or a mixture, and typically releases a bronchodilator in a time scale of seconds and minutes.

Specifically, the steroid is released between an hour to 15 days of application to a human being, while the first bronchodilator molecule is released between 1 seconds to 30 minutes of application.

Specifically, the therapeutic concentration of bronchodilator is reached within 10 seconds of administration and persists for 10 hours. The therapeutic efficacy of the corticosteroid is reached within 2 hours and can persist for 15 days.

Preferably the rapidly-degrading polymers are synthesized through ester, carboxyl or amine linkages, and the degradation is triggered following exposure to enzymes or a pathophysiological condition inside the body.

Preferably the rapidly-degrading polymers or the one or all the lipids in the mixture of lipids used to synthesize the outer layer of the nanocell is modified chemically to evade the immune system, such as through pegylation or the addition of polyethylene chains. They can also be modified for increasing hydrophilicity on the surface.

### (ii) Cancer

In some embodiments, the nanocell core is constructed of biodegradable polymers and releases an antineoplastic agent in a time scale of days; in some embodiments, the nanocell matrix surrounds the core and is constructed out of rapidly-degrading polymer or lipid mixture, and releases an antiangiogenesis or a vascular targeting agent at a time scale of hours.

Preferably, the antineoplastic agent is released between 2 hours to 15 days of application to a human being, while the antiangiogenesis molecule is released between 10 min to 72 hours of application, preferably between 30 min and 56 hours of administration.

Specifically, the active concentration of the antiangiogenesis agent is reached within 5 hours, while the therapeutic concentration of the antineoplastic agent is reached within 1 days.

Preferably the rapidly-degrading polymers are synthesized through ester, carboxyl or amine linkages, and the degradation is triggered following exposure to enzymes or a pathophysiological condition inside the body.

Preferably the rapidly-degrading polymers or the one or all the lipids in the mixture of lipids used to synthesize the outer layer of the nanocell is modified chemically to evade the immune system, such as through pegylation or the addition of polyethylene chains. They can also be modified for increasing hydrophilicity on the surface.

(iii) Arthritis

In some embodiments, nanocells for use in the treatment of arthritis have a core constructed of biodegradable polymers that release a corticosteroid or a DMARD in a time scale of days. Desirably, such nanocells may also have a matrix surrounding the nanocore that is constructed out of rapidly-degrading polymer or a mixture of lipids, and that releases an antiangiogenesis or a vascular targeting agent at a time scale of hours.

Preferably such rapidly-degrading polymers are synthesized through ester, carboxyl or amine linkages, and the degradation is triggered following exposure to enzymes or a pathophysiological condition inside the body.

Preferably the rapidly-degrading polymers or the one or all the lipids in the mixture of lipids used to synthesize the outer layer of the nanocell is modified chemically to evade the immune system, such as through pegylation or the addition of polyethylene chains. They can also be modified for increasing hydrophilicity on the surface.

Preferably, the corticosteroid or the DMARD agent is released between 2 hours to 15 days of application to a human being, while the antiangiogenesis molecule is released between 10 min to 72 hours of application, preferably between 30 min and 56 hours of administration.

Specifically, the active concentration of the antiangiogenesis agent is reached within 5 hours, while the therapeutic concentration of the corticosteroid/DMARD agent inside the diseased site is reached within 1 days.

### (iv) Multiple Sclerosis

In some embodiments, nanocells for use in the treatment of multiple sclerosis have a core constructed of biodegradable polymers that releases a corticosteroid or a disease modifying agent in a time scale of days. Desirably, such nanocells may also have a matrix surrounding the nanocore that is constructed out of rapidly-degrading polymer or a mixture of lipids, and that releases an antiangiogenesis or a vascular targeting agent at a time scale of hours.

Preferably the rapidly-degrading polymers are synthesized through ester, carboxyl or amine linkages, and the degradation is triggered following exposure to enzymes or a pathophysiological condition inside the body.

Preferably the rapidly-degrading polymers or the one or all the lipids in the mixture of lipids used to synthesize the outer layer of the nanocell is modified chemically to evade the immune system, such as through pegylation or the addition of polyethylene chains. They can also be modified for increasing hydrophilicity on the surface.

Specifically, the active concentration of the antiangiogenesis agent is reached within 5 hours, while the therapeutic concentration of the corticosteroid agent is reached within 1 days.

Preferably, the corticosteroid or the disease modifying agent is released between 2 hours to 15 days of application to a human being, while the antiangiogenesis molecule is released between 10 min to 72 hours of application, preferably between 30 min and 56 hours of administration.

Specifically, the active concentration of the antiangiogenesis agent is reached within 5 hours, while the therapeutic concentration of the corticosteroid or disease modifying agent agent inside the diseased site is reached within 1 days.

### (v) Psoriasis

In some embodiments, nanocells for use in the treatment of psoriasis have a core constructed of biodegradable polymers that releases a corticosteroid or a disease modifying agent in a time scale of days. In some embodiments, the core is surrounded by a matrix, constructed out of rapidly-degrading polymer or a mixture of lipids, that releases an antiangiogenesis or a vascular targeting agent at a time scale of hours.

Preferably the rapidly-degrading polymers are synthesized through ester, carboxyl or amine linkages, and the degradation is triggered following exposure to enzymes or a pathophysiological condition inside the body.

Preferably the rapidly-degrading polymers or the one or all the lipids in the mixture of lipids used to synthesize the outer layer of the nanocell is modified chemically to evade the immune system, such as through pegylation or the addition of polyethylene chains. They can also be modified for increasing hydrophilicity on the surface.

Preferably, the corticosteroid or the disease modifying agent is released between 2 hours to 15 days of application to a human being, while the antiangiogenesis molecule is released between 10 min to 72 hours of application, preferably between 30 min and 56 hours of administration.

Specifically, the active concentration of the antiangiogenesis agent is reached within 5 hours, while the therapeutic concentration of the corticosteroid or disease modifying agent agent inside the diseased site is reached within 1 days.

The present invention also provides a gel formulation with embedded nanocells for treatment of psoriasis. Such a gel formulation may desirably be applied topically.

### (vi) Sports Injuries

In some embodiments, the present invention provides nanocells for the treatment of muscle injuries. For example, in such embodiments, an outer layer may desirably encapsulate a fast acting muscle relaxant and the nanocore may encapsulate an NSAID.

In some embodiments, the present invention provides nanocells for the treatment of sports injuries. For example, in such embodiments, an outer layer may encapsulate a fast acting muscle relaxant and the nanocore may encapsulate an NSAID.

In some embodiments of the invention, a formulation for sports injuries is administered topically as an aerosol or spray, for example in which a muscle relaxant is released immediately on contact with body surface, and the NSAID is slowly released from the nanoparticle.

In some embodiments of an inventive nanocell formulation for sports injuries, the formulation is administered topically as an aerosol or spray, and the muscle relaxant is released immediately from the outer surface of the nanocell on contact with body surface while the NSAID is slowly released from the nanocore.

In some embodiments of an inventive nanocell formulation for sports injuries, the formulation is administered topically as an aerosol or spray, and the muscle relaxant is released in a time scale of seconds to minutes from the outer surface of the nanocell on contact with body surface, while the NSAID is slowly released from the nanocore on a time scale of minutes to hours.

In some embodiments of an inventive nanocell formulation for sports injuries, the formulation is administered topically as an aerosol or spray, and the muscle relaxant is released and absorbed in a time scale of seconds to minutes from the outer surface of the nanocell on contact with body surface. In some such embodiments, the nanocore penetrates the skin and slowly releases the NSAIDs in a slow release manner leading to increased focal concentrations and less systemic absorption.

In some embodiments of an inventive nanocell formulation for sports injuries, the formulation is administered topically as an aerosol or spray, and the muscle relaxant is released preferably in a time scale of 15 seconds to 30 min from the outer surface of the nanocell on contact with body surface, while the NSAID is slowly released from the nanocore in a time scale of 3 min to 24 hours.

In some embodiments of an inventive nanocell formulation for sports injuries, the formulation is administered topically as an aerosol or spray, and about 50% of the muscle relaxant is released before about 25% of the NSAID is released from the nanocore.

It is a further object of the current invention to provide an assay system that allows the screening of anti-angiogenic agents and chemotherapeutic agents together or separately in a situation similar to an in vivo environment. This includes cells growing on extra-cellular matrix, and accurately simulates in vivo condition. In this assay, the endothelial cells are seeded and allowed to grow on the extracellular matrix before the tumor cells are seeded on the tissue culture plate. To detect the tumor cells, they are transfected to express a fluorescent gene product such as green fluorescent protein (GFP). The endothelial cells are stained with a fluorescent dye. Kits with the necessary agents need to practice the inventive assay method are also provided by the present invention.

### DEFINITIONS

“Adjuvant”: The term adjuvant refers to any compound which is a nonspecific modulator of the immune response. In certain preferred embodiments, the adjuvant stimulates the immune response. Any adjuvant may be used in accordance with the present invention. A large number of adjuvant compounds is known; a useful compendium of many such compounds is prepared by the National Institutes of Health (see also Allison Dev. Biol. Stand. 92:3-11, 1998; Unkeless et al. Annu Rev. Immunol. 6:251-281, 1998; and Phillips et al. Vaccine 10:151-158, 1992, each of which is incorporated herein by reference).

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Antibody”: The term antibody refers to an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in the present invention. Itinerary

“Antibody fragment”: The term antibody fragment refers to any derivative of an antibody which is less than full-length. Preferably, the antibody fragment retains at least a significant protion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

Diabodies are dimeric scFvs. The components of diabodies typically have shorter peptide linkers than most scFvs, and they show a preference for associating as dimers.

An Fv fragment is an antibody fragment which consists of one V_{H} and one V_{L} domain held together by noncovalent interactions. The term dsFv is used herein to refer to an Fv with an engineered intermolecular disulfide bond to stabilize the V_{H}-V_{L} pair.

A F(ab′)₂ fragment is an antibody fragment essentially equivalent to that obtained from immunoglobulins (typically IgG) by digestion with an enzyme pepsin at pH 4.0-4.5. The fragment may be recombinantly produced.

A Fab fragment is an antibody fragment essentially equivalent to that obtained by reduction of the disulfide bridge or bridges joining the two heavy chain pieces in the F(ab′)₂ fragment. The Fab′ fragment may be recombinantly produced.

A Fab fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 30%, 20%, 10%, 5%, or 1% cell death and do not induce inflammation or other such unwanted adverse effects in vivo.

“Biodegradable”: As used herein, “biodegradable” compounds are those that, when introduced into cells, are broken down by the cellular machinery into components that the cells can either reuse or dispose of without significant toxic effect on the cells (i.e., fewer than about 30%, 20%, 10%, 5%, or 1% of the cells are killed).

“Effective amount”: In general, the “effective amount” of an active agent or the microparticles refers to the amount necessary to elicit the desired biological response. As will be appreciated by those of ordinary skill in this art, the effective amount of microparticles may vary depending on such factors as the desired biological endpoint, the agent to be delivered, the composition of the encapsulating matrix, the target tissue, etc. For example, the effective amount of microparticles containing an anti-epileptic agent to be delivered is the amount that results in a reduction in the severity or frequency of seizures and/or unwanted electrical activity. In another example, the effective amount of microparticles containing an anti-arrhythmic medication to be delivered to the heart of the individual is the amount that results in a decrease in the amount or frequency of the unwanted electrical activity, or decrease in clinical signs (e.g., ECG findings) or symptoms (e.g., syncopal episodes) of cardiac arrhythmias.

“Nanocell”: According to the present invention, the term “nanocell” refers to a particle in which a nanocore is surrounded or encapsulated in a matrix or shell. In other words, a smaller particle within a larger particle, or a balloon within a balloon. The nanocell preferably has an agent in the nanocore, and a different agent in the outer portion of the nanocell. In certain preferred embodiments, the nanocell is a nanocore inside a liposome. In other embodiments, the nanocore is surrounded by a polymeric matrix or shell (e.g., a polysaccharide matrix).

“Nanocore”: As used herein, the term “nanocore” refers to any particle within a nanocell. A nanocore may be a microparticle, a nanoparticle, a quantum dot, a nanodevice, a nanotube, a nanoshell, or any other composition of the appropriate dimensions to be included within a nanocell. Preferably, the nanocore comprises an agent to be released more slowly or after the agent in the outer portion of the nanocell is released.

The term “nasal delivery” refers to delivery of nanocells by inhalation through and into the nose.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “pulmonary delivery” and “respiratory delivery” refer to delivery of nanocells to a patient by inhalation through the mouth and into the lungs.

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

### BRIEF DESCRIPTION OF THE DRAWING

FIG. 1 is a schematic of a nanocell particle. The nanocells includes a nanocore loaded with a first agent inside a lipid vesicle enclosing a second agent.

FIG. 2 shows an alternative combination therapy strategy. A targeted nanoparticle with a first agent is used in conjunction with a unilamellar lipid vesicle containing a second agent to achieve the slow and fast pharmacokinetics of the nanocell.

FIG. 3 shows the synthesis and characterization of a combretastatin-doxorubicin nanocell. (A) Schematic of conjugation reactions between doxorubicin and PLGA 5050. (B) The scanning electron micrograph (Jeol JSM5600, 3700x) of nanoparticles synthesized using an emulsion-solvent evaporation technique shows the spherical structures of heterogenous sizes. (C) Structure of combretastatin, which is encapsulated in the lipid bilayer. (D) Transmission electron microscopy image of the cross section of three nanocells, obtained by sectioning at a thickness of 70 nm, staining with 2.0% uranyl acetate followed by 0.1% lead citrate and examining using a Philips EM410. With this technique, the nanoparticle (dark sphere) appears nuclear surrounded by a white crown of phospholipid block co-polymers. (E) Sizing using dynamic laser light scatter demonstrate that nanoparticles of the defined size could be isolated through sequential steps of ultracentrifugation, for encapsulation in phospholipid copolymer envelope. (F) Physicochemical release-rate kinetics profile for combretastatin and doxorubicin shows that combretastatin is released first from the nanocell followed by free doxorubicin. Dexamethasone was used as the internal standard. Data shown are mean±SE with n=4. Data points where error bars are not visible means the error is small and hidden by the plot. ***P<0.002; #P<0.001 vs combretastatin concentration at same time points.

FIG. 4 shows the effects of VEGF and HGF on tumor angiogenesis in vitro, and the effect of PTK787, aVEGF-receptor antagonist.

FIG. 5 shows the effect of doxorubicin, thalidomide, and combretastatin on VEGF-induced response in a co-culture assay of B16/F10 melanoma cells and human umbilical vein endothelial cells.

FIG. 6 shows the effect of doxorubicin, thalidomide, and combretastatin on HGF-induced response in a co-culture assay of B16/F10 melanoma cells and human umbilical vein endothelial cells.

FIG. 7 shows the effect of doxorubicin, thalidomide, and combretastatin on VEGF-induced response in a co-culture assay of B16/F10 melanoma cells and human umbilical vein endothelial cells, when plated on collagen.

FIG. 8 shows the effect of doxorubicin, thalidomide, and combretastatin on HGF-induced response in a co-culture assay of B16/F10 melanoma cells and human umbilical vein endothelial cells, when plated on collagen.

FIGS. 9A-9H show a bioassay of the temporal release and activity of pharmacological agents from the nanocell. A GFP+melanoma-endothelial cell coculture was established on a 3-dimensional matrigel matrix. The co-culture was incubated with different treatment groups for defined time periods. Cells were fixed with paraformaldehyde, stained with propidium iodide, and analysed using a Zeiss LSM510 confocal microscope. Fluorochromes were excited with 488 nm and 543 nm laser lines, and the images were captured using 505-530 BP and 565-615 BP filters at a 512×512 pixel resolution. The micrographs depict merge images from different treatment groups (FIGS. 9A-9F). The melanoma cells appear yellow while the vessel forming endothelial cells are red in color. The graph (FIG. 9G) depicts the stereological quantification of the area covered by each cell type. Treatment with nanocells (NC) result in a temporal rapid ablation of the vasculature followed by delayed loss of the tumor cells. In contrast, control groups treated with liposomal-combretastatin (250 μg/ml) (L[C]) or doxorubicin-conjugated nanoparticles (ND) (20 μg/ml of Doxorubicin) resulted in selective loss of vasculature or tumor cell respectively. The image for 30 h NC treatment was specifically selected to show a few rounded cells to emphasise the ablation of the co-culture, although complete cell loss was evident in most images. Four random images were captured from each replicate in an experiment. Data represents mean±SEM from 3 independent experiments. The concentration-effect curve (FIG. 9H) shows the effect of free doxorubicin and PLGA-conjugated doxorubicin on B16/F10 cells. [Dox] indicates the concentration of drug added to the culture as free drug or in nanocells. Data shown are mean±SE of 2 independent experiments with replicates. ***P<0.001 (ANOVA with Bonferroni's post-hoc test).

FIGS. 10A-10Q demonstrate that nano cell therapy inhibits B16/F10 melanoma and Lewis lung carcinoma growth. Melanoma and carcinoma were established in C57/BL6 mice following the subcutaneous injection of 3×10⁵ GFP+BL6/F10 or 2.5×10⁵ Lewis lung carcinoma cells into the flanks Excised tumors showing the effects of nanocells (NC) (FIGS. 10F and 10L) vs. the effects of nanocells with only doxorubicin-conjugated nanoparticles NC[D] (FIGS. 10B and 10H), liposomal-combretastatin (L[C]) (FIGS. 10D and 10J), the co-injection of NC[D]+L[C] (FIGS. 10C and 10I), a simple liposomal formulation encapsulating both combretastatin and doxorubicin (L[CD]) (FIGS. 10E and 10K), and a lower dose (Id) of NC (FIG. 10M). Control groups were treated with saline. Carcinoma and melanoma (50 mm³)-bearing mice were randomised into 6-8 groups, and treated every alternate day with the different vehicles equivalent to 50 mg/kg and 500 μg/kg of combretastatin and doxorubicin respectively. Graphs (FIGS. 10N and 10O) show the mean (SE) tumor volume in different treatment groups, calculated from the measurement of the longest and the shortest diameter of carcinoma and melanoma. The graphs (FIG. 10P) show the effect of different treatments on the white blood cell counts. The least toxicity was observed with the nanocell-treated group. Long-term treatment with nanocells (NClt) had no additional toxicity as compared to the shorter treatment. In FIG. 10Q, the distribution of nanocells, fabricated with fluorescein dye, was quantified over time by measuring the levels of the dye at 5, 10 and 24 hours. At 24 hours, a preferential accumulation of the nanocells in the carcinoma was evident in comparison to other vascularised tissues, with a concomitant fall of the levels in blood. All data are mean±SEM with n=3-5 per group depending upon the time points. Data points where error bars are not visible means the error is small and hidden by the plot.

FIGS. 11A-11T show the effect of nanocell treatment on tumor vasculature and apoptosis. Tumors were excised from Lewis lung carcinoma-bearing animals treated with nanocells (NC) (FIGS. 11E and 11K), nanocells with only doxorubicin-conjugated nanoparticles NC[D] (FIGS. 11C and 11I), liposomal-combretastatin (L[C]) (FIGS. 11B and 11H), the co-injection of NC[D]+L[C] (FIGS. 11D and 11J), or a simple liposomal formulation encapsulating both combretastatin and doxorubicin (L[CD]) (FIGS. 11F and 11L). Control groups recieved saline. Treatment was administered every alternate day over the 10 day period, using the different vehicles equivalent to 50 mg/kg and 500 μg/kg of combretastatin and doxorubicin respectively. The top panel (FIGS. 11A-11F) shows the cross-section of tumors, fixed with cold methanol, and immunostained for von Willebrand factor (vWF), a vascular endothelial marker. The lower panel (FIGS. 11G-11L) shows the effect of different treatments on the induction of apoptosis in the tumors. The sections were fixed in 10% formalin, and processed for TUNEL+positive staining using Texas red labeled nucleotide. The same sections were co-labeled with an antibody against HIF-1α, and detected using a FITC-labeled secondary antibody. The yellow signal in the merged image in the NC-treated group demonstrates the nuclear localization of HIF-1α as the TUNEL staining detects DNA strand-breaks, a hallmark of apoptosis. The graphs depict the (FIG. 11M) tumor vessel density, (FIG. 11N) % of hypoxic cells, and (FIG. 11O) % of apoptotic cells, calculated applying standard stereology techniques to tumor cross sections. All images were captured using a Zeiss LSM510 confocal microscope. The fluorochromes were excited with 488 nm and 543 nm laser lines, and the images were captured using 505-530 BP and 565-615 BP filters at a 512×512 pixel resolution. Data are expressed as mean±SEM from three independent tumor samples, with multiple random images from each sample. *P<0.05, **P<0.01, ***P<0.001 vs controls (ANOVA with Newman-Keul's Post Hoc test). The western blots (FIGS. 11P-11R) show the effect of different treatments on the levels of HIF1α (FIG. 11Q) and VEGF (FIG. 11P), and is quantitatively normalized to β-actin (FIG. 11R) in graphs (FIG. 11S & FIG. 11T) respectively. *P<0.05 vs. other combretastatin-treated groups.

FIGS. 12A-12B show the effect of liposomal and nanocell combretastatin and long-term nanocell therapy on tumor growth. (FIG. 12A) Graph shows the effect of liposomal combretastatin and nanocells (fabricated encapsulating only combretastatin and PLGA core) were administered to melanoma-bearing mice. Treatment was started when the tumors reached 50 mm3 in volume and continued every alternate day for five rounds of administration. The total combretastatin administered per injection in either formulation was 50 mg/kg. In another experiment, melanoma-bearing animals were treated with seven cycles of NC therapy once the tumors reached 50 mm³ in volume. Control animals were treated with PBS vehicle, and were sacrificed on day 17 as the tumors became too big in size. In the long-term treated group, 50% of the animals showed almost complete regression of tumor over 28 days, and as shown in graph (FIG. 12B) the remaining animals had significantly smaller tumor volume as compared to the untreated animals.

FIGS. 13A-13N show the effect of nanocell therapy on metastasis of primary GFP+melanoma to lungs and liver. The upper panel (FIGS. 13A-13F) depicts a cross-section of same-level lung tissues from different treatment groups. The lower panel (FIGS. 13G-13L) shows the same level cross-sections of livers from different treatment groups. The organs were excised from animals treated with nanocells (NC), doxorubicin-conjugated nanoparticles NC [D], liposomal-combretastatin (L[C]), or co-injected with NC[D]+LC, or doxorubicin and combretastatin encapsulated liposomes (L[CD]). Control groups were treated with saline. The tissues were fixed in 4% paraformaldehyde on ice, and stained with standard H&E. The images were captured using a Zeiss LSM510 confocal microscope. The fluorochromes were excited with 488 nm and 543 nm laser lines, and the images were captured using 505-530 BP and 565-615 BP filters at a 512×512 pixel resolution. The merge images shown here demonstrate distinct metastatic nodes, which appear yellow. The graphs depict the quantification of metastatic nodes in each view field, included the lungs (FIG. 13M and liver (FIG. 13N). Data expressed are mean±SEM from n=3. ***P<0.001 vs. controls (ANOVA with Newman-Keul's Post Hoc test).

FIG. 14 is a schematic showing the detailed synthetic steps involved in the conjugation of doxorubicin to PLGA 5050.

FIG. 15 shows the structure and release kinetic profile of nanocells developed for treatment of asthma. The electron micrograph shows the ultrastructure of the outer matrix of these nanocells where the matrix is a lactose shell. A corticosteroid (anti-inflammatory agent) can be entrapped within the nanocore, while a bronchodilator is entrapped in the lactose matrix surrounding the nanocore. The graphs demonstrate the fact that the bronchodilator (salbutamol) is released first in a time scale of minutes, while the corticosteroid (dexamethasone) is released in a slow prolonged manner. This temporal release would enable the constricted bronchioles during asthma to get dilated first allowing the permeation of the nanocores into deeper lung. The subsequent slow release would block the chronic inflammation that follows an acute asthma episode while the fast release of salbutamol alleviates immediate symptoms.

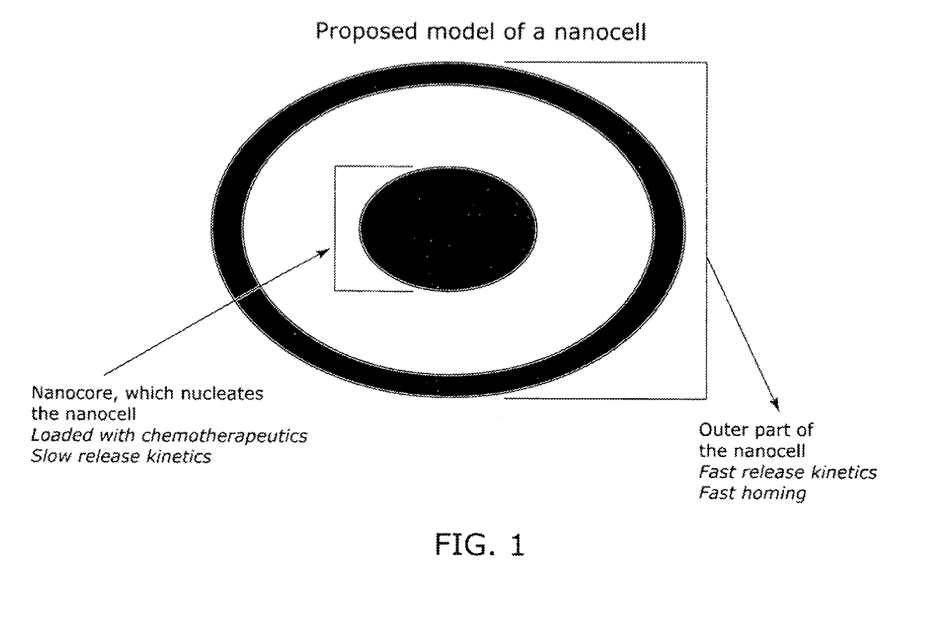

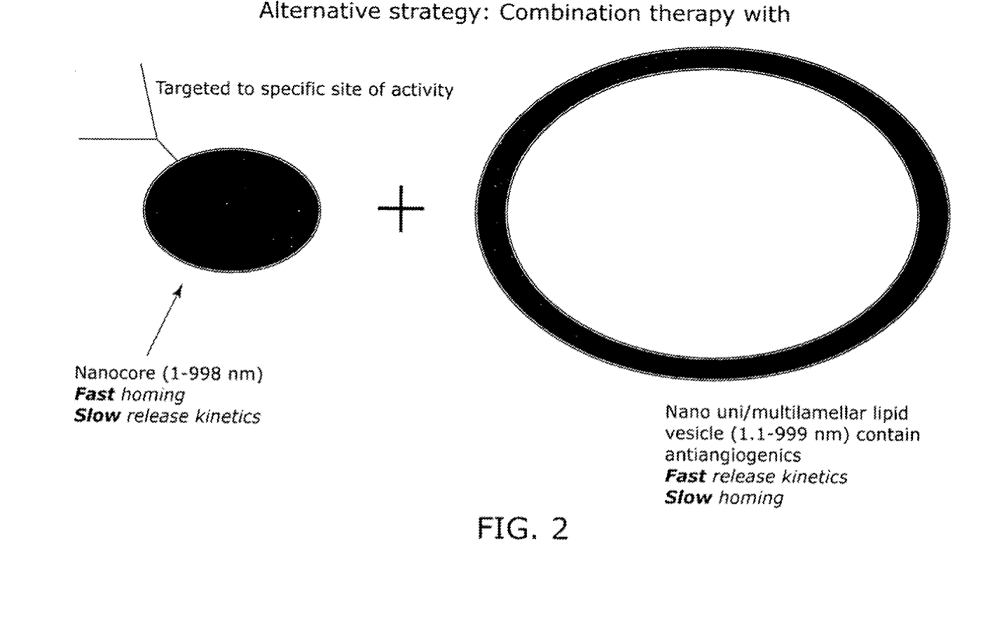

### DETAILED DESCRIPTION OF CERTAIN PREFERRED EMBODIMENTS OF THE INVENTION

The inventive drug delivery system stems from the recognition that in administering multiple agents to treat a disease, it may be advantageous to deliver one agent or combination of agents before a second agent or set of agents is delivered. The agents being released at different times using the inventive particles may have different modes of action, different targets, and/or different pharmacokinetic profiles. The present invention includes the inventive particles (nanocells), pharmaceutical compositions with nanocells, methods of preparing nanocells and pharmaceutical compositions thereof, and method of using nanocells and pharmaceutical compositions thereof. A nanocell is conceptually a balloon within a balloon or a particle (e.g., a nanoparticle) within a particle (e.g., liposome).

In one embodiment, a nanocell includes an inner portion (nanocore) loaded with a first agent or combination of agents surrounded by a lipid vesicle or matrix/shell outer portion with a second agent or combination of agents. The agent(s) in the outer portion is released before the agent(s) in the inner nanocore. Preferably, a nanocell contains one nanocore. In certain embodiments, however, a nanocell contains between one or multiple nanocores, preferably between one and one hundred nanocores, more preferably between one and ten nanocores, and even more preferably between one and three nanocores. In another embodiment, a nanocell is a particle with an inner core coated with an outer shell or matrix.

The core of the inventive nanocells includes at least one agent encapsulated in a matrix. The matrix is preferably a polymeric matrix that is biodegradable and biocompatible. Polymers useful in preparing the nanocore include synthetic polymers and natural polymers. Examples of polymers useful in the present invention include polyesters, polyamides, polyethers, polythioethers, polyureas, polycarbonates, polycarbamides, proteins, polysaccharides, polyaryls, etc. The polymers useful in the nancores have average molecular weights ranging from 100 g/mol to 100,000 g/mol, preferably 500 g/mol to 80,000 g/mol. In a preferred embodiment, the polymer is a polyester synthesized from monomers selected from the group consisting of D,L-lactide, D-lactide, L-lactide, D, L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid. More preferably, the biodegradable polyester is synthesized from monomers selected from the group consisting of D,L-lactide, D-lactide, L-lactide, D, L-lactic acid, D-lactic acid, L-lactic acid, glycolide, glycolic acid, ε-caprolactone, and ε-hydroxy hexanoic acid. Most preferably, the biodegradable polyester is synthesized from monomers selected from the group consisting of D,L-lactide, D-lactide, L-lactide, D, L-lactic acid, D-lactic acid, L-lactic acid, glycolide, and glycolic acid. Copolymers may also be used in the nanocore. Copolymers include ABA-type triblock copolymers, BAB-type triblock copolymers, and AB-type diblock copolymers. The block copolymers may have hydrophobic A blocks (e.g., polyesters) and hydrophilic B block (e.g., polyethylene glycol).

The polymer of the nanocore is chosen based on the entrapment and release kinetics of the active agent. In certain embodiments, the active agent on the nanocore is covalently linked to the polymer of the nanocore. To covalently link the agent to be delivered to the polymer matrix, the polymer may be chemically activated using any technique known in the art. The activated polymer is then mixed with the agent under suitable conditions to allow a covalent bond to form between the polymer and the agent. In preferred embodiments, a nucleophile, such as a thiol, hydroxyl group, or amino group, on the agent attacks an electrophile (e.g., activated carbonyl group) on the polymer to create a covalent bond.

In other embodiments, the active agent is associated with the matrix of the nanocore through non-covalent interactions such as van der Waals interactions, hydrophobic interactions, hydrogen bonding, dipole-dipole interactions, ionic interactions, and pi stacking

The nanocores may be prepared using any method known in the art for preparing nanoparticles. Such methods include spray drying, emulsion-solvent evaporation, double emulsion, and phase inversion. In addition, any nanoscale particle, matrix, or core may be used as the nanocore inside a nanocell. The nanocore may be, but are not limited, to nanoshells (see U.S. Pat. No. 6,685,986, incorporated herein by reference); nanowires (see U.S. Pat. No. 5,858,862, incorporated herein by reference); nanocrystals (see U.S. Pat. No. 6,114,038, incorporated herein by reference); quantum dots (see U.S. Pat. No. 6,326,144, incorporated herein by reference); and nanotubes (see U.S. Pat. No. 6,528,020, incorporated herein by reference).

After the nanocores are prepared, they may be fractionated by filtering, sieving, extrusion, or ultracentrifugation to recover nanocores within a specific size range. One effective sizing method involves extruding an aqueous suspension of the nanocores through a series of polycarbonate membranes having a selected uniform pore size; the pore size of the membrane will correspond roughly with the largest size of nanocores produced by extrusion through that membrane. See, e.g., U.S. Pat. No. 4,737,323, incorporated herein by reference. Another preferred method is serial ultracentrifugation at defined speeds (e.g., 8,000, 10,000, 12,000, 15,000, 20,000, 22,000, and 25,000 rpm) to isolate fractions of defined sizes. In certain embodiments, the nanocores are prepared to be substantially homogeneous in size within a selected size range. The nanocores are preferably in the range from 10 nm to 10,000 nm in their greatest diameter. More preferably, the nanocores range from 20 to 8,000 nm in their greatest diameter, most preferably from 50 to 5,000 nm in their greatest diameter. The nanocores may be analyzed by dynamic light scattering and/or scanning electron microscopy to determine the size of the particles. The nanocores may also be tested for loading the agent(s) into the nanocore. Nanocores include nanoparticles as well as nanoshells, nanowire, quantum dots, and nanotubes.

Once the nanocores have been prepared and optionally characterized, the nanocores are coated with an outer layer such as a lipid, polymer, carbohydrate, etc. to form a nanocell. The nanocores may be coated with a synthetic or naturally occurring macromolecule, such as a lipid, carbohydrate, polysaccharide, protein, polymer, glycoproteins, glycolipids, etc. using any method described in the art. Various methods of preparing lipid vesicles have been described including U.S. Pat. Nos. 4,235,871, 4,501,728, 4,837,028; PCT Application WO 96/14057, New RRC, Liposomes. A practical approach, IRL Press, Oxford (1990), pages 33-104; Lasic D D, Liposomes from physics to applications, Elsevier Science Publishers BV, Amsterdam, 1993; Szoka et al., Ann. Rev. Biophys. Bioeng. 9:467 (1980); Liposomes, Marc J. Ostro, ed., Marcel Dekker, Inc., New York, 1983, Chapter 1; Hope et al., Chem. Phys. Lip. 40:89 (1986); each of which is incorporated herein by reference.

Any lipid including surfactants and emulsifiers known in the art is suitable for use in making the inventive nanocells. The lipid component may also be a mixture of different lipid molecules. These lipid may be extracted and purified from a natural source or may be prepared synthetically in a laboratory. In a preferred embodiment, the lipids are commercially available. Lipids useful in coating the nanocores include natural as well as synthetic lipids. The lipids may be chemically or biologically altered. Lipids useful in preparing the inventive nanocells include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; and phospholipids. The lipid may be positively charged, negatively charged, or neutral. In certain embodiments, the lipid is a combination of lipids. Phospholipids useful in preparing nanocells include negatively charged phosphatidyl inositol, phosphatidyl serine, phosphatidyl glycerol, phosphatic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitotylphosphatidyl glycerol, distearyloylphosphatidyl glycerol, dimyristoyl phosphatic acid, dipalmitoyl phosphatic acid, dimyristoyl phosphitadyl serine, dipalmitoyl phosphatidyl serine, phosphatidyl serine, and mixtures thereof. Useful zwitterionic phospholipids include phosphatidyl choline, phosphatidyl ethanolamine, sphingomyeline, lecithin, lysolecithin, lysophatidylethanolamine, cerebrosides, dimyristoylphosphatidyl choline, dipalmitotylphosphatidyl choline, distearyloylphosphatidyl choline, dielaidoylphosphatidyl choline, dioleoylphosphatidyl choline, dilauryloylphosphatidyl choline, 1-myristoyl-2-palmitoyl phosphatidyl choline, 1-palmitoyl-2-myristoyl phosphatidyl choline, 1-palmitoyl-phosphatidyl choline, 1-stearoyl-2-palmitoyl phosphatidyl choline, dimyristoyl phosphatidyl ethanolamine, dipalmitoyl phosphatidyl ethanolamine, brain sphingomyelin, dipalmitoyl sphingomyelin, distearoyl sphingomyelin, and mixtures thereof. Zwitterionic phospholipids constitute any phospholipid with ionizable groups where the net charge is zero. In certain embodiments, the lipid is phosphatidyl choline.

Cholesterol and other sterols may also be incorporated into the lipid outer portion of the nanocell of the present invention in order to alter the physical properties of the lipid vesicle. utable sterols for incorporation in the nanocell include cholesterol, cholesterol derivatives, cholesteryl esters, vitamin D, phytosterols, ergosterol, steroid hormones, and mixtures thereof. Useful cholesterol derivatives include cholesterol-phosphocholine, cholesterolpolyethylene glycol, and cholesterol-SO₄, while the phytosterols may be sitosterol, campesterol, and stigmasterol. Salt forms of organic acid derivatives of sterols, as described in U.S. Pat. No. 4,891,208, which is incorporated herein by reference, may also be used in the inventive nanocells.

The lipid vesicle portion of the nanocells may be multilamellar or unilamellar. In certain embodiments, the nanocore is coated with a multilamellar lipid membrane such as a lipid bilayer. In other embodiments, the nanocore is coated with a unilamellar lipid membrane.

Derivatized lipids may also be used in the nanocells. Addition of derivatized lipids alter the pharmacokinetics of the nanocells. For example, the addition of derivatized lipids with a targeting agent may allow the nanocells to target a specific cell, tumor, tissue, organ, or organ system. In certain embodiments, the derivatized lipid components of nanocells include a labile lipid-polymer linkage, such as a peptide, amide, ether, ester, or disulfide linkage, which can be cleaved under selective physiological conditions, such as in the presence of peptidase or esterase enzymes or reducing agents. Use of such linkages to couple polymers to phospholipids allows the attainment of high blood levels for several hours after administration, else it may be subject to rapid uptake by the RES system. See, e.g., U.S. Pat. No. 5,356,633, incorporated herein by reference. The pharmacokinetics and/or targeting of the nanocell can also be modified by altering the surface charge resulting from changing the lipid composition and ratio. Thermal or pH release characteristics can be built into nanocell by incorporating thermal sensitive or pH sensitive lipids as a component of the lipid vesicle (e.g., dipalmitoyl-phosphatidylcholine:distearyl phosphatidylcholine (DPPC:DSPC) based mixtures). Use of thermal or pH sensitive lipids allows controlled degradation of the lipid vesicle membrane component of the nanocell.

Additionally, the nanocell according to the present invention may contain non-polymeric molecules bound to the exterior, such as haptens, enzymes, antibodies or antibody fragments, cytokines, receptors, and hormones (see, e.g., U.S. Pat. No. 5,527,528, incorporated herein by reference), and other small proteins, polypeptides, or non-protein molecules which confer a particular enzymatic or surface recognition feature to lipid formulations. Techniques for coupling surface molecules to lipids are known in the art (see, e.g., U.S. Pat. No. 4,762,915, incorporated herein by reference).

In one embodiment, the lipids are dissolved in a suitable organic solvent or solvent system and dried under vacuum or an inert gas to form a thin lipid film. Optionally, the film may be redissolved in a suitable solvent, such as tertiary butanol, and then lyophilized to form a more homogeneous lipid mixture, which is in a more easily hydrated powder-like form. The resulting film or powder is covered with an aqueous buffered suspension of nanocores and allowed to hydrate over a 15-60 minute period with agitation. The size distribution of the resulting multilamellar vesicles can be shifted toward smaller sizes by hydrating the lipids under more vigorous agitation conditions or by adding a solubilizing detergent such as deoxycholate.

In another embodiment, the coating of the nanocore may be prepared by diffusing a lipid-derivatized with a hydrophilic polymer into pre-formed vesicles, such as by exposing pre-formed vesicles to nanocores/micelles composed of lipid-grafted polymers at lipid concentrations corresponding to the final mole percent of derviatized lipid which is desired in the nanocell. The matrix, surrounding the nanocore, containing a hydrophilic polymer can also be formed by homogenization, lipid-field hydration, or extrusion techniques.

In yet another embodiment, the nanocores are first dispersed by sonication in a low CMC surfactant, such as lysophosphatidylcholine, including polymer-grafted lipids that readily solubilizes hydrophobic molecules. The resulting micellar suspension of nanocores is then used to rehydrate a dried lipid sample that contains a suitable mole percent of polymer-grafter lipid, or cholesterol. The matrix/shell and nanocore suspension is then formed into nanocells using extrusion techniques known in the art. The resulting nanocells are separated from the unencapsulated nanocores by standard column chromatography.

In another preferred embodiment, vesicle-forming lipids are taken up in a suitable organic solvent or solvent system, and dried or lyophilized in vacuo or under an inert gas to form a lipid film. The active agent/s that is/are to be incorporated in the outer chamber of the nanocell, are preferably included in the lipids forming the film. The concentration of drug in the lipid solution may be included in molar excess of the final maximum concentration of drug in the nanocells, to yield maximum drug entrapment in the nanocells. The aqueous medium used in hydrating the dried lipid or lipid/drug is a physiologically compatible medium, preferably a pyrogen-free physiological saline or 5% dextrose in water, as used for parenteral fluid replacement. The nanocores are suspended in this aqueous medium in a homogenous manner, and at a desired concentration of the other active agent/agents in the nanocore, prior to the hydration step. The solution can also be mixed with any additional solute components, such as a water-soluble iron chelator, and/or a soluble secondary compound at a desired solute concentration. The lipids are allowed to hydrate under rapid conditions (using agitation) or slow conditions (without agitation). The lipids hydrate to form a suspension of multilamellar vesicles whose size range is typically between about 0.5 microns to 10 microns or greater. In general, the size distribution of the vesicles can be shifted toward smaller sizes by hydrating the lipid film more rapidly while shaking. The structure of the resulting membrane bilayer is such that the hydrophobic (non-polar) “tails” of the lipid orient toward the center of the bilayer, while the hydrophilic (polar) “heads” orient towards the aqueous phase.

In another embodiment, dried vesicle-forming lipids, agent-containing nanocores, and the agent(s) (to be loaded in the outer chamber of the nanocell) mixed in the appropriate ratios, are dissolved, with warming if necessary, in a water-miscible organic solvent or mixture of solvents. Examples of such solvents are ethanol, or ethanol and dimethylsulfoxide (DMSO) in varying ratios. The mixture then is added to a sufficient volume of an aqueous receptor phase to cause spontaneous formation of nanocells. The aqueous receptor phase may be warmed if necessary to maintain all lipids in the melted state. The receptor phase may be stirred rapidly or agitated gently. The mixture may be injected rapidly through a small orifice, or poured in directly. After incubation of several minutes to several hours, the organic solvents are removed, by reduced pressure, dialysis, or diafiltration, leaving a nanocell suspension suitable for human administration.

In another embodiment, dried vesicle-forming lipids, the agent/s to be loaded in the outer chamber of the nanocell, and the agent-loaded nanocore mixed in the appropriate amounts are dissolved, with warming if necessary, in a suitable organic solvent with a vapor pressure and freezing point sufficiently high to allow removal by freeze-drying (lyophilization). Examples of such solvents are tert-butanol and benzene. The drug/lipid/solvent mixture then is frozen and placed under high vacuum. Examples of methods for freezing include “shell-freezing,” in which the container containing the mixture is swirled or spun to maximize contact of the liquid with the walls of the vessel, and the container is placed in a cooled substance such as liquid nitrogen or carbon dioxide ice mixed with a solvent such as an alcohol or acetone. The mixture thus is frozen rapidly without segregation of the constituents of the drug/lipid/solvent mixture. A fluffy, dry powder results from removal of the solvent by lyophilization. This drug/lipid powder may be stored for extended periods under conditions that reduce chemical degradation of the constituents or the absorption of moisture. Examples of such conditions include sealing the powder under an atmosphere of dry, inert gas (such as argon or nitrogen), and storage in the cold. When it is desired to administer the material, reconstitution is performed by adding a physiologically compatible aqueous medium, preferably a pyrogen-free physiological saline or 5% dextrose in water. If the second active agent/s is/are hydrophilic, it can also be added at this stage. Reconstitution causes the spontaneous formation of nanocells, which may be refined in size by methods detailed herein including ultracentrifugation, filtering, and sieving.

As would be appreciated by one of skill in this art, any pharmaceutical, diagnostic, or prophylactic agent may be administered using the inventive drug delivery system. The agents being loaded into the two compartments of the nanocells will depend of various factors including the disease being treated, the patient, the clinical setting, the mode of administration, and other factors that would be appreciated by one of ordinary skill in the art such as a licensed physician or pharmacologist.

In certain embodiments, the agent in the nanocore, the inner portion of the nanocell, has slower release kinetics than the agent in the outer portion of the nanocell. In this way, the agent in the outer portion is released first and is allowed to exert its effect before the agent in the nanocore begins to exerts its effect. For example, in treating cancer, the outer lipid vesicle portion of the nanocell is load with a traditional chemotherapeutic agent such as methotrexate, and the nanocore is loaded with an antiangiogenesis agent such as combretastatin. Methotrexate is released first from the nanocells, and the blood supply to the tumor carries the cytotoxic agent to the tumor cells before combretastatin cuts off the blood supply to the tumor. In this way, the cytotoxic agent is allowed to get to the cells and exert its cytotoxic effect before the anti-angiogenic agent cuts off the blood supply to the tumor. The sequential delivery of a cytotoxic agent followed by an antiangiogenic agent is preferably synergistic allowing for decreased side effects due to the lower doses of drugs being used in the inventive system.

Agents being delivery using the inventive nanocells include therapeutic, diagnostic, or prophylactic agents. Any chemical compound to be administered to an individual may be delivered using nanocells. The agent may be a small molecule, organometallic compound, nucleic acid, protein, peptide, metal, an isotopically labeled chemical compound, drug, vaccine, immunological agent, etc.

In a preferred embodiment, the agents are organic compounds with pharmaceutical activity. In another embodiment of the invention, the agent is a clinically used drug. In another embodiment, the agent has been approved by the U.S. Food & Drug Administration for use in humans or other animals. In a particularly preferred embodiment, the drug is an antibiotic, anti-viral agent, anesthetic, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, nutritional agent, etc. For example, inventive nanocells may be prepared so that they include one or more compounds selected from the group consisting of drugs that act at synaptic and neuroeffector junctional sites (e.g., acetylcholine, methacholine, pilocarpine, atropine, scopolamine, physostigmine, succinylcholine, epinephrine, norepinephrine, dopamine, dobutamine, isoproterenol, albuterol, propranolol, serotonin); drugs that act on the central nervous system (e.g., clonazepam, diazepam, lorazepam, benzocaine, bupivacaine, lidocaine, tetracaine, ropivacaine, amitriptyline, fluoxetine, paroxetine, valproic acid, carbamazepine, bromocriptine, morphine, fentanyl, naltrexone, naloxone); drugs that modulate inflammatory responses (e.g., aspirin, indomethacin, ibuprofen, naproxen, steroids, cromolyn sodium, theophylline); drugs that affect renal and/or cardiovascular function (e.g., furosemide, thiazide, amiloride, spironolactone, captopril, enalapril, lisinopril, diltiazem, nifedipine, verapamil, digoxin, isordil, dobutamine, lidocaine, quinidine, adenosine, digitalis, mevastatin, lovastatin, simvastatin, mevalonate); drugs that affect gastrointestinal function (e.g., omeprazole, sucralfate); antibiotics (e.g., tetracycline, clindamycin, amphotericin B, quinine, methicillin, vancomycin, penicillin G, amoxicillin, gentamicin, erythromycin, ciprofloxacin, doxycycline, acyclovir, zidovudine (AZT), ddC, ddI, ribavirin, cefaclor, cephalexin, streptomycin, gentamicin, tobramycin, chloramphenicol, isoniazid, fluconazole, amantadine, interferon); anti-cancer agents (e.g., cyclophosphamide, methotrexate, fluorouracil, cytarabine, mercaptopurine, vinblastine, vincristine, doxorubicin, bleomycin, mitomycin C, hydroxyurea, prednisone, tamoxifen, cisplatin, decarbazine); immunomodulatory agents (e.g., interleukins, interferons, GM-CSF, TNFα, TNFβ, cyclosporine, FK506, azathioprine, steroids); drugs acting on the blood and/or the blood-forming organs (e.g., interleukins, G-CSF, GM-CSF, erythropoietin, vitamins, iron, copper, vitamin B₁₂, folic acid, heparin, warfarin, coumarin); hormones (e.g., growth hormone (GH), prolactin, luteinizing hormone, TSH, ACTH, insulin, FSH, CG, somatostatin, estrogens, androgens, progesterone, gonadotropin-releasing hormone (GnRH), thyroxine, triiodothyronine); hormone antagonists; agents affecting calcification and bone turnover (e.g., calcium, phosphate, parathyroid hormone (PTH), vitamin D, bisphosphonates, calcitonin, fluoride), vitamins (e.g., riboflavin, nicotinic acid, pyridoxine, pantothenic acid, biotin, choline, inositol, carnitine, vitamin C, vitamin A, vitamin E, vitamin K), gene therapy agents (e.g., viral vectors, nucleic-acid-bearing liposomes, DNA-protein conjugates, anti-sense agents); or other agents such as targeting agents etc.

Prophylactic agents include vaccines. Vaccines may comprise isolated proteins or peptides, inactivated organisms and viruses, dead organisms and virus, genetically altered organisms or viruses, and cell extracts. Prophylactic agents may be combined with interleukins, interferon, cytokines, and adjuvants such as cholera toxin, alum, Freund's adjuvant, etc. Prophylactic agents include antigens of bacteria, viruses, fungi, protozoa, and parasites. These antigens may be in the form of whole killed organisms, peptides, proteins, glycoproteins, carbohydrates, or combinations thereof.

Agent may mean a combination of agents that have been combined and loaded into the nanocore or outer lipid portion of the nanocell. Any combination of agents may be used. For example, pharmaceutical agents may be combined with diagnostic agents, pharmaceutical agents may be combined with prophylactic agents, pharmaceutical agents may be combined with other pharmaceutical agents, diagnostic agents may be combined with prophylactic agents, diagnostic agents may be combined with other diagnostic agents, and prophylactic agents may be combined with other prophylactic agents. In certain embodiments for treating cancer, at least two traditional chemotherapeutic agents are loaded into the other lipid portion of a nanocell.

In one aspect of the present invention, the nanocells are prepared to have substantially homogeneous sizes in a selected size range. The nanocells may be filtered, sieved, centrifuged, ultracentrifuged, sorted by column chromatography, or extruded to collect particles of a particular size. One effective sizing method involves extruding an aqueous suspension of the nanocells through a series of polycarbonate membranes having a selected uniform pore size; the pore size of the membrane will correspond roughly with the largest sizes of nanocells produced by extrusion through that membrane. See, e.g., U.S. Pat. No. 4,737,323, incorporated herein by reference. Another preferred method is by serial ultracentrifugation at defined speeds to isolate fractions of defined sizes.

Although, a preferred use of the nanocell composition would be in tumor therapy, both solid and myeloid, the same principle is embodied in the treatment of other abnormal angiogenesis-based pathologies. Other pathologies may include arthritis, retinopathies, psoriasis, solid tumors, benign tumors, Kaposi's sarcoma, and hematological malignancies. This could include drugs described earlier; or for example in the case of arthritis, it may comprise of disease modifying drugs (DMARDs), non-steroidal anti-inflammatory drugs (NSAIDS), Colchicine, methotrexate, etc. in the nanocore with an anti-angiogenic agent in the surrounding lipid vesicle or polymeric shell. In addition, the spatiotemporal release kinetics and pharmacodynamic synergism between two unrelated active agents achieved with the nanocell opens up the possibility of its use in other pathophysiological conditions where such a temporal or spatial activity of therapeutic agents is desired. Examples of such conditions could be asthma, where a antispasmodic or relaxant drug is loaded in the outer portion of the nanoshell while an anti-inflammatory agent, such as a steroid or NSAID, is loaded in the nanocore for delayed activity against the delayed inflammatory reaction associated with asthma, and would exert its effect after the fast released active agent from the outer portion of the nanocell has relaxed the alveoli and/or bronchioles. Similarly, molecules that open up the blood brain barrier can be loaded in the outer portion of the nanocell while centrally acting neuroactive agents can be loaded into the nanocore, resulting in a increase build-up of the active agent in the CNS. Nanocells can also be used in the delivery of vaccines for a better outcome. For example, an inflammatory agent such as an adjuvant may be loaded into the outer portion of the nanocell, and an antigen loaded into the nanocore. As would be appreciated by one of skill in this art, the nanocell system may be used to treat a wide variety of diseases.

The present invention encompasses novel nanocell platforms for the treatment of various diseases and disorders. In addition, methods for the treatment of specific diseases and disorders utilizing these compositions are disclosed. Nanocells (see U.S. patent application Ser. No. 11/070,731, filed Mar. 2, 2005) can be tailored so that they directly and efficiently deliver appropriate therapies for appropriate lengths of time to relevant biological sites.

In general, tailored nanocells of the present invention comprise an inner nanocore containing at least one first therapeutic and at least one outer nanoshell comprised of lipid, which contains at least one second therapeutic that differs from the first therapeutic. Alternatively, the nanocore may contain at least one therapeutic that is substantially similar to the at least one therapeutic contained in the nanoshell. In this embodiment, the composition of the matrix encapsulating the first therapeutic differs from the composition of the matrix encapsulating the at least one second therapeutic so that the therapies are released a different times and/or rates. One can also add third, fourth, fifth, or more layers designed to release the same or different agent at specified times.

In one embodiment of the present invention, a novel composition and method for treating a desired angiogenic disease or disorder, e.g. tumors, is disclosed. In this embodiment, the nanocell comprises a nanocore containing a first therapeutic that is selectively chosen so as to act over an extended period of time and a second therapeutic encapsulated within the outer nanoshell that is selectively chosen so as to act immediately and over a shorter period of time. In one preferred embodiment the tailored nanocells are size restricted such as being greater than about 60 nm so that they selectively extravasate at sites of angiogenesis (e.g. tumor, macular degeneration) and do not pass through normal vasculature or enter non-tumor bearing tissue. In a preferred embodiment of the present invention, the tailored nanocell is about 60 nm to about 120 nm in total diameter.

In one embodiment the first therapeutic, located in the nanocore, is an anti-neoplastic and the second therapeutic, located in the nanoshell is an anti-angiogenic.

Anti-neoplastic compounds include, but are not limited to, compounds such as floxuridine, gemcitabine, cladribine, dacarbazine, melphalan, mercaptopurine, thioguanine, cisplatin, and cytarabine; and anti-viral compounds such as fludarabine, cidofovir, tenofovir, and pentostatin. Further examples of compounds suitable for association with the nanocore include adenocard, adriamycin, allopurinol, alprostadil, amifostine, aminohippurate, argatroban, benztropine, bortezomib, busulfan, calcitriol, carboplatin, daunorubicin, dexamethasone, topotecan, docetaxel, dolasetron, doxorubicin, epirubicin, estradiol, famotidine, foscarnet, flumazenil, fosphenyloin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard.

Anti-angiogenic compounds include, but are not limited to anti-VEGF antibodies, including humanized and chimeric antibodies, anti-VEGF aptamers and antisense oligonucleotides, angiostatin, endostatin, interferons, interleukin 1, interleukin 12, retinoic acid, and tissue inhibitors of metalloproteinase-1 and -2.

In one embodiment the tailored nanocell for the treatment of angiogenic diseases and disorders is specific for lung cancer. In this embodiment, the first therapeutic, located in the nanocore, is selected from the group consisting of cisplatin, carboplatin, Iressa, or Gefitinib and the second therapeutic is a corticosteroid. In this embodiment, the nanocell is greater than about 60 nm.

In another embodiment, the tailored nanocell for the treatment of angiogenic diseases and disorders is specific for breast or kidney cancer. In this embodiment, the first therapeutic in doxorubicin and the second therapeutic is a corticosteroid. In this embodiment, the nanocell is greater than about 60 nm.

In another embodiment, the tailored nanocell for the treatment of angiogenic diseases and disorders is specific for skin cancer and/or melanoma. In this embodiment, the first therapeutic in dacarbazine (DTIC) and the second therapeutic is a corticosteroid. In this embodiment, the nanocell is greater than about 60 nm.

In another embodiment, the tailored nanocell for the treatment of angiogenic diseases and disorders is specific for GI tumors. In this embodiment, the first therapeutic is 5-fluorouracil (5-FU) and the second therapeutic is a corticosteroid. In this embodiment, the nanocell is greater than about 60 nm.

As used herein, the term “corticosteroid” refers to any of the adrenal corticosteroid hormones isolated from the adrenal cortex or produced synthetically, and derivatives thereof that are used for treatment of inflammatory diseases, such as arthritis, asthma, psoriasis, inflammatory bowel disease, lupus, and others. Corticosteroids include those that are naturally occurring, synthetic, or semi-synthetic in origin, and are characterized by the presence of a steroid nucleus of four fused rings, e.g., as found in cholesterol, dihydroxycholesterol, stigmasterol, and lanosterol structures. Corticosteroid drugs include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-11β,17-dihydroxy-16α-methylpregna-1,-4-diene-3,20-dione), and highly derivatized steroid drugs such as beconase (beclomethasone dipropionate, which is 9-chloro-11β, 17,21, trihydroxy-16β-methylpregna-1,4diene-3,20-dione 17,21-dipropionate). Other examples of corticosteroids include flunisolide, prednisone, prednisolone, methylprednisolone, triamcinolone, deflazacort and betamethasone.

(i) Brain Tumor

In one embodiment, a composition and method for the treatment of brain tumors, such as, for example, gliomas, neuronal tumors, anaplastic glioma and meningioma is disclosed. Other brain tumors treatable by the methods and compositions of the present invention include, but are not limited to, astrocytomas, brain stem gliomas, ependymomas, oligodendogliomas, and non-glial originated brain tumors such as medulloblastomas, meningiomas, Schwannomas, craniopharyngiomas, germ cell tumors, pineal region tumors, and secondary brain tumors.

In this embodiment, the nanocell composition comprises a nanocore with at least one first therapeutic consisting of a corticosteroid and a nanoshell with at least one second therapeutic consisting of a chemotherapeutic. As used herein, a chemotherapeutic includes any cancer treatment, such as, chemical agents or drugs, that are selectively destructive to malignant cells and tissues. The corticosteroid may be selected from the group consisting of cortisol, cortisone, hydrocortisone, fludrocortisone, prednisone, methylprednisonlone, prednisolone or the like. Other corticosteroids are known to those of skill in the art and encompassed in the present invention.

The chemotherapeutic, located in the nanoshell may be selected from the group consisting of nitrosurea-based chemotherapy such as, for example, BCNU (carmustine), CCNU (lomustine), PCV (procarbazine, CCNU, vincristine), or temozolomide (Temodar). Other chemotherapeutics are known to those of skill in the art and may be used in the methods of the present invention. They include, for example, alkylating agents, antitumor antibiotics, plant alkaloids, antimetabolites, hormonal agonists and antagonists, and a variety of miscellaneous agents. See Haskell, C. M., ed., (1995) and Dorr, R. T. and Von Hoff, D. D., eds. (1994). The classic alkylating agents are highly reactive compounds that have the ability to substitute alkyl groups for the hydrogen atoms of certain organic compounds. The classic alkylating agents include mechlorethamine, chlorambucil, melphalan, cyclophosphamide, ifosfamide, thiotepa and busulfan. A number of nonclassic alkylating agents also damage DNA and proteins, but through diverse and complex mechanisms, such as methylation or chloroethylation, that differ from the classic alkylators. The nonclassic alkylating agents include dacarbazine, carmustine, lomustine, cisplatin, carboplatin, procarbazine and altretamine.

Clinically useful antitumor drugs include natural products of various strains of the soil fungus Streptomyces, which are also encompassed in the present invention. Drugs of this class include doxorubicin (Adriamycin), daunorubicin, idarubicin, mitoxantrone, bleomycin, dactinomycin, mitomycin C, plicamycin and streptozocin. Plants-based chemotherapies are also encompassed and include the Vinca alkaloids (vincristine and vinblastine), the epipodophyllotoxins (etoposide and teniposide) and paclitaxel (Taxol). In addition, antimetabolites such as methotrexate, 5-fluorouracil (5-FU), floxuridine (FUDR), cytarubine, 6-mercaptopurine (6-MP), 6-thioguanine, deoxycoformycin, fludarabine, 2-chlorodeoxyadenosine, and hydroxyurea are also encompassed in the present invention.

Preferably, the first therapeutic is encapsulated in a biodegradable polymer, so as to provide for sustained or slow-release kinetics of the corticosteroid. The chemotherapeutic is also encapsulated in biodegradable polymer, so as to provide for a more immediate release of a specific agent. The ratio may be tailored so as to tailor treatment to an individual, rather than the current method of same treatment for every individual. For example, Roche's AmpliChip CYP450®, which analyzes an individuals metabolism toward certain drugs may be used to assess the optimal dose required for a particular individual. In this way, a practioner is able to combine appropriate nanocores (with optimal PHA ratios) with optimal nanoshells to achieve optimal dosing.

Also encompassed in the present invention are methods for the treatment of brain tumors utilizing the tailored nanocell composition of the invention. In this method, an individual is administered a tailored nanocell of the present invention systemically or by directly injecting into the site in need. Preferably, the tumor is resected and the tailored nanocells are delivered to the area of resection at this time.

Therefore, in further aspects of the present invention, the nanocell compositions described herein may be used for the treatment of angiogenic diseases and disorders and malignancy. Within such methods, the nanocell compositions described herein are administered to a patient, typically a warm-blooded animal, preferably a human. A patient may or may not be afflicted with cancer. Accordingly, the above nanocell compositions may be used to prevent the development of a cancer or to treat a patient afflicted with a cancer. Tailored nanocell compositions may be administered either prior to or following surgical removal of primary tumors and/or treatment such as administration of radiotherapy or conventional chemotherapeutic drugs. Administration of the nanocell compositions may be by any suitable method, including administration by intravenous, intraperitoneal, intramuscular, subcutaneous, intranasal, intradermal, anal, vaginal, topical and oral routes.

(ii) Asthma

In another embodiment, a composition and method for the treatment of asthma is disclosed. In this embodiment, the nanocell composition comprises a nanocore with at least one first therapeutic consisting of a corticosteroid and a nanoshell with at least one second therapeutic consisting of a bronchodilator. The corticosteroid may be selected from the group consisting of cortisol, cortisone, hydrocortisone, fludrocortisone, prednisone, methylprednisonlone, or prednisolone etc. The bronchodilator may include an anticholinergic, such as ipratropium or a beta-agonist such as albuterol, metaproterenol, pirbuterol, or levalbuteral. The nanocell composition for the treatment of asthma allows for an individual to be administered a smaller dose of corticosteroid than is normally attainable due to the administration of the bronchodilator (encased in the nanoshell), which acts first to make available the biological sites of action for the corticosteroid.

Alternatively, anti-IgE may be incorporated into the nanocore of the nanocell alone or in addition to a corticosteroid. Anti-IgE therapy is a long-term therapy and thus should be formulated in the nanocore of the present composition so as to sustain delivery over time. Commercially available anti-IgE includes Xolairg (omalizumab), which is approved for individuals with moderate to severe persistent asthma, year round allergies and who are taking routine inhaled steroids.

In another embodiment, the tailored-asthma nanocell may comprise Intal® (cromolyn) and/or Tilade® (nedocromil), which help prevent asthma symptoms, especially symptoms caused by exercise, cold air and allergies. Cromolyn and nedocromil help prevent swelling in airways. Because cromolyn and nedocromil are preventive, and must be taken on a regular basis to be effective, they are best suited for incorporation into the nanocore of the asthma-tailored nanocell.

In another embodiment, the tailored asthma nanocell contains leukotriene modifiers such as, for example, Accolate® (zafirlukast), Singulair® (montelukast), and Zyflo® (zileuton). Leukotriene modifiers may be incorporated into either the nanocore or nanoshell, but preferably into the nanocore where they act over an extended period of time. Leukotriene modifiers may be incorporated into the nanocell alone or in addition to other therapies.

Although one can use any method to deliver the nanocell, it is preferred that the asthma tailored nanocell is delivered via inhalation.

(iii) Grave's Disease

In another embodiment, a composition and method for the treatment of Grave's Disease is disclosed. In this embodiment, the nanocell composition comprises a nanocore with at least one first therapeutic consisting of iopanoic acid/ipodate sodium and a nanoshell with at least one second therapeutic consisting of an antithyroid drug such as, for example, methimazole, carbimazole, or propylthiouracil. Alternatively, the first therapeutic may be a radioiodine, such as iodine 123. In one embodiment the nanocore comprises radioiodine alone or in combination with iopanoic acid/ipodate sodium. Likewise, the at least one second therapeutic, incorporated in the nanoshell, may be a beta-blocker (i.e. propanolol).

Other beta-blockers useful in the present invention include acebutolol, atenolol, betaxolol, bisoprolol, carteolol, labetalol, metoprolol, nadolol, oxprenolol, penbutolol, pindolol, sotalol, timolol, atenolol,

Preferably, a tailored nanocell of the present invention is delivered systemically via parenteral or enteral routes.

(iv) Cystic Fibrosis

In another embodiment, a composition and method for the treatment of Cystic Fibrosis is disclosed. In this embodiment, the nanocell composition comprises a nanocore with at least one first therapeutic consisting of an antibiotic. In addition to an antibiotic, the core may also contain an optional bronchodilator or steroid. In this embodiment, the nanoshell contains at least one second therapeutic consisting of recombinant human deoxyribonuclease (rhDNase).

Antibiotics are known to those of skill in the art. See, for example, Curr Opin Pulm Med. November 2004; 10(6):515-23; Ann Pharmacother. January 2005; 39(1):86-94; Respir Med. January 2005; 99(1):1-10. Preferred antibiotics include, but are not limited to ciprofloxacin, ofloxacin, tobramycin (including TOBI), gentamicin, azithromycin, ceftazidime, Keflex® (cephalexin), Ceclor® (cefaclor), piperacillin and imipenem.

In another embodiment, the tailored cystic fibrosis nanocell comprises S-nitrosothiol in a form suitable for administration to a CF patient and formulated to maximize contact with epithelial surfaces of the respiratory tract. S-Nitrosoglutathione is the most abundant of several endogenous S-nitrosothiols. It is uniquely stable compared, for example, to 5-nitrosocysteine unless specific GSNO catabolic enzymes are upregulated. Such enzymes can include gamma-glutamyl-transpeptidase, glutathione-dependent formaldehyde dehydrogenase, and thioredoxin-thioredoxin reductase. For this reason, co-administration of inhibitors of GSNO prokaryotic or eukaryotic GSNO catabolism may at times be necessary and are encompassed in the present invention. This kind of inhibitor would include, but not be limited to, acivicin given as 0.05 ml/kg of a 1 mM solution to achieve an airway concentration of 1 μM S-nitrosoglutathione (GSNO). Preferably, the S-nitrosoglutathione (GSNO) is in concentrations equal to or in excess of 500 nmole/kg (175 mcg/kg). Other nitrosylating agents such as ethyl nitrite may also be used. Thus, the methods and compositions of the present invention comprise a nitrosonium donor including, but not limited to GSNO and other S-nitrosothiols (SNOs) in a pharmaceutically acceptable carrier that allows for administration by nebulized or other aerosol treatment to patients with cystic fibrosis. These compounds may be incorporated into either the nanocore or nanoshell of the cystic fibrosis nanocell of the present invention.

Preferably, an individual is administered a tailored nanocell of the present invention via inhalation.

(v) Pulmonary Fibrosis

In another embodiment, a composition and method for the treatment of pulmonary fibrosis is disclosed. Pulmonary fibrosis may also be termed Idiopathic Pulmonary Fibrosis, Interstitial Pulmonary Fibrosis, DIP (Desquamative interstitial pneumonitis), UID (Usual interstitial pneumonitis), all of which are encompassed in the present invention. In this embodiment, the nanocell composition comprises a nanocore with at least one first therapeutic consisting of an antiflibrotic agent such as colchine (also known as colchicines) and a nanoshell with at least one second therapeutic consisting of a corticosteroid, such as, for example, cortisol, cortisone, hydrocortisone, fludrocortisone, prednisone, methylprednisonlone, or prednisolone etc. The antifibrotic agent may also be selected from the group consisting of Pirfenidone (Deskar; MARNAC, Inc., Dallas, Tex.), colchicine, D-penicillamine, and interferon.

Preferably, an individual is administered a tailored nanocell of the present invention via inhalation.

Some corticosteroids useful for this invention include, but are not limited to, cortisol, cortisone, hydrocortisone fludrocortisone, prednisone, prednisolone, 6-methylprednisolone, triamcinolone, betamethasone, and dexamethasone. However, any of the adrenal corticosteroid hormones isolated from the adrenal cortex or produced synthetically, and derivatives thereof that are used for treatment of inflammation are useful for this invention.

The tailored nanocells of the present invention may contain more than two layers. In one embodiment, the tailored nanocell comprises a plurality of reservoirs where drugs are deposited in layers. Optionally, polymer membranes may be positioned in between the drug-polymer layers for controlled release of various drugs.

In general, the tailored nanocells of the present invention may be administered to individuals as described above, but may also be administered in manner known to those of skill in the art and so as to tailor administration to an individuals needs. For example, dosage may be adjusted appropriately to achieve a desired therapeutic effect. It will be understood that the specific dose level and frequency of dosage for any particular subject may be varied and will depend upon a variety of factors including the activity of the specific therapeutically active agent employed, the metabolic stability and length of action of that agent, the species, age, body weight, general health, dietary status, sex and diet of the subject, the mode and time of administration, rate of excretion, drug combination, and severity of the particular condition. Generally, daily doses of active therapeutically active agents can be determined by one of ordinary skill in the art without undue experimentation, in one or several administrations per day, to yield the desired results.

In the event that the response in a subject is insufficient at a certain dose, even higher doses (or effective higher doses by a different, more localized delivery route) may be employed to the extent that patient tolerance permits. Multiple doses per day are contemplated to achieve appropriate systemic or targeted levels of therapeutic compounds.

### Targeting Agents

The nanocells may be modified to include targeting agents since it is often desirable to target a drug delivery device to a particular cell, collection of cells, tissue, or organ. A variety of targeting agents that direct pharmaceutical compositions to particular cells are known in the art (see, for example, Cotten et al. Methods Enzym. 217:618, 1993; incorporated herein by reference). The targeting agents may be included throughout the nanocells, only in the inner nanocore, only in the outer lipid or polymeric shell portion, or may be only on the surface of the nanocell. The targeting agent may be a protein, peptide, carbohydrate, glycoprotein, lipid, small molecule, metal, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, etc. If the targeting agent is included in the nanocore, the targeting agent may be included in the mixture that is used to form the nanoparticles. If the targeting agent is only on the surface of the nanocells, the targeting agent may be associated with (i.e., by covalent, hydrophobic, hydrogen boding, van der Waals, or other interactions) the formed particles using standard chemical techniques.

### Pharmaceutical Compositions

Once the inventive particles have been prepared, they may be combined with other pharmaceutical excipients to form a pharmaceutical composition. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), transdermally, subcutaneously, bucally, or as an oral or nasal spray.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

(i) Respirable Nanocells

One aspect of the invention provides aerosols for the delivery of nanocells to the respiratory tract. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung.

Herein, administration by inhalation may be oral and/or nasal. Examples of pharmaceutical devices for aerosol delivery include metered dose inhalers (MDIs), dry powder inhalers (DPIs), and air jet nebulizers.

The human lungs can remove or rapidly degrade hydrolytically cleavable deposited aerosols over periods ranging from minutes to hours. In the upper airways, ciliated epithelia contribute to the “mucociliary excalator” by which particles ate swept from the airways toward the mouth. Pavia, D., “LungMuicociliary Clearance,” in Aerosols and the Lung: Clinical and Experimental Aspects, Clarke, S. W. and Pavia, D., Eds., Butterworths, London, 1984. In the deep lungs, alveolar macrophages are capable of phagocytosing particles soon after their deposition. Warheit et al. Microscopy Res. Tech., 26: 412-422 (1993); and Brain, J. D., “Physiology and Pathophysiology of Pulmonary Macrophages,” in The Reticuloendothelial System, S. M. Reichard and J. Filkins, Eds., Plenum, New. York., pp. 315-327, 1985. The deep lung, or alveoli, are the primary target of inhaled therapeutic aerosols for systemic delivery of nanocells.

### Method for Assaying a Pharmaceutical Composition

Intervention of the parenchyma-stroma axis remains an attractive goal for tumor therapy. Standard approaches to evaluate anti-angiogenics have been to study its activity on endothelial cell proliferation, migrations, chemoinvasion or tubulogenesis, which are the key steps during angiogenesis (Sengupta et al., Circulation 107(23):2955-61, Jun. 17, 2003). However, these assays are limited by the fact that the activated endothelium is studied in isolation from tumor cells. This is vital since tumor endothelium has been demonstrated to exhibit unique genetic signatures (StCroix et al. Science 289(5482):1197-1202, Aug. 18, 2000). Furthermore, standard tissue culture techniques often do not promote spatial arrangements. Indeed, endothelial cells grown in 2-D systems vary from 3-D model systems that have been developed to simulate natural interactions between cells and the extracellular environment. Shekhar et al. (Cancer Res. 61(4):1320-26, Feb. 15, 2001) developed a 3-dimensional matrigel-based co-culture model, where endothelial cells mixed with pre-neoplastic breast epithelial cells allowed the study of ductal-alveolar morphogenesis, angiogenesis, and progression to malignant phenotype. Nehls and Drenckhahn (Histochem. Cell Biol. 104(6):459-66, December 1995) used a micro-carrier-based fibrin gel-embedded co-culture, while Dutt et al. (Tissue Eng. 9(5):893-908, October 2003) used a NASA Bioreactor to develop a 3D co-culture system. Longo et al. (Blood 98(13):3717-26, Dec. 15, 2001) studied the interactions of melanoma cells with a monolayer of endothelial cells on a 3-D collagen matrix. However, in all such co-culture experiments, endothelial cells are labeled using commonly used antibodies such as CD31, CD34, CD105, vWF, etc., or lectins that bind to al-fucosyl moieties, using standard immunohistocytochemistry, which is costly and time intensive. Furthermore, the simultaneous visualization and analysis of the interacting cell partners adds another level of complexity.

The current invention overcomes these limitations, as it incorporates stably transfected the transformed tumor cells (e.g., melanoma cells) to express a fluorescent gene product (e.g., green fluorescent protein (GFP)), without altering the primary endothelial cell that has a finite lifetime. The subsequent one-step labeling of the endothelial and tumor components distinctly, allows easy visualization and analysis since a merged image depicts the tumor cell in a color different that the endothelial cells (e.g., the tumor cell as green, while the endothelial cells appear red).

Indeed, the incubation with doxorubicin exerted a chemotherapeutic effect as evident from the complete loss of the green melanoma cells. Furthermore, the capture of high contrast images with lower background also facilitated stereological analysis for quantification, a step that can easily be computationally automated.

The cell lines used in the assay system are any transformed cell that can stably express a fluorescent protein or has been modified to fluoresce when excited using an appropriate wavelength. Preferably, the cells would be from a tumor of mesenchymal origin (sarcomas), or from a tumor of epithelial origin (carcinomas), or a teratoma. Cells from brain cancer, lung cancer, stomach cancer, colon cancers, breast cancers, bladder cancers, prostate cancer, ovarian cancers, uterine cancers, testicular cancers, pancreatic cancers, leukemias, lymphomas, bone cancers, muscle cancers, and skin cancers may be used in the inventive assay. Preferably, the cells would be adherent to a cell culture dish. Endothelial cells should be from the vascular system, e.g., arteries, veins, or the microvasculature such as the capillaries. The endothelial cells can be derived from progenitor cells or stem cells. In certain embodiments, the endothelial cells are derived from human umbilical cords.

In all co-culture experiments reported prior to this study, the interacting cellular components were seeded together. However, in pathophysiology, angiogenesis is defined as the sprouting of neovasculature from an existing vascular bed. To mimic the pathophysiology more accurately, the current invention allows the development of primordial networks of endothelial cells to form, prior to seeding the tumor cells. A significant increase in the formation of vascular networks in the presence of tumor cells is observed following this approach. This novel in vitro model system simulates tumor angiogenesis more accurately, and allows the simultaneous detection of chemotherapeutic and anti-angiogenic activity of novel molecules. This assay system will provide an unique tool to dissect out the molecular interactions of the parenchyma-stroma axis, and facilitate the development of strategic combination regimens of chemotherapeutics and anti-angiogenics.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Synthesis and Analysis of Nanocells

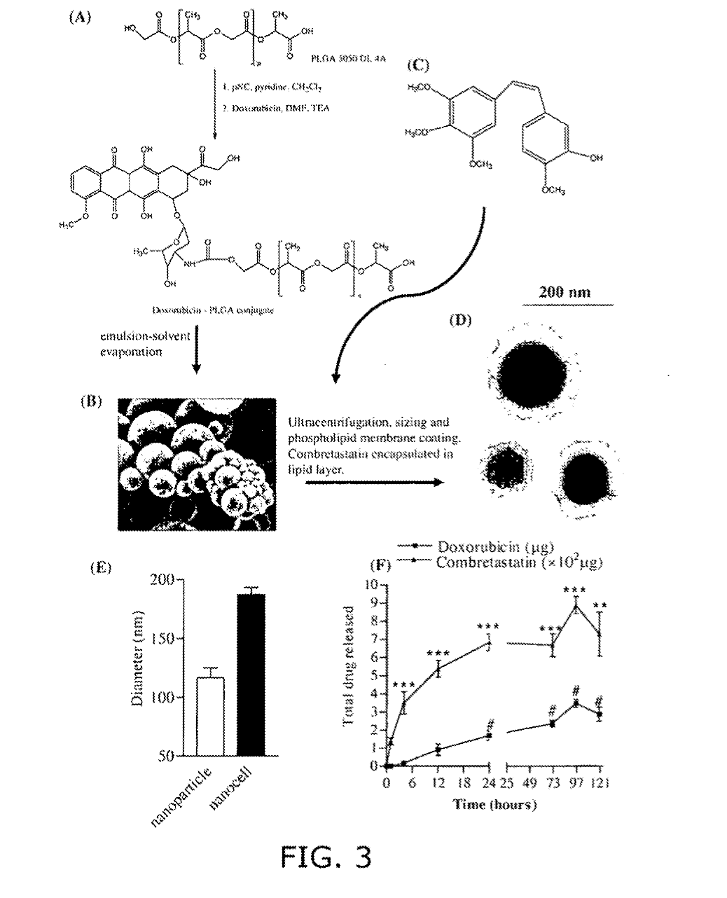

(A) Conjugation of Doxorubicin to PLGA (FIG. 3). Polylactic glycolic acid (PLGA) (Medisorb® 5050 DL 4A), having a lactide/glycolide molar ratio of 50/50, was obtained from Alkermes (Wilmington, Ohio). The average molecular weight of this polymer is reported to be 61 kDa, and it has free hydroxyl and carboxylic groups at its terminal ends. Doxorubicin hydrochloride, p-nitrophenyl chloroformate, and triethylamine were obtained from Sigma-Aldrich (St. Louis, Mo.). Briefly, 1.5 g of PLGA 5050 DL 4A was dissolved in 15 ml of methylene chloride and activated by the addition of 14 mg of p-nitrophenyl chloroformate and 9.4 mg (˜9.6 μL) of pyridine to the solution, kept in an ice bath at 0° C. (stoichiometric molar ratio of PLGA:p-nitrophenyl chloroformate:pyridine=1:2.8:4.7). The reaction was carried out for 3 hours at room temperature under nitrogen atmosphere. The resulting solution was diluted with methylene chloride and washed with 0.1% HCl and brine solution. The organic phase was separated, dried on anhydrous magnesium sulfate, filtered, and then rotary-evaporated to yield activated PLGA polymer. Activated PLGA (0.4 g) was dissolved in 3 mL of dimethylformamide (DMF) and reacted with 4 mg of doxorubicin and 2.7 mg (˜4 μL) of triethylamine for 24 hours at room temperature under nitrogen atmosphere (stoichiometric molar ratio of activated PLGA:doxorubicin:triethylamine=1:1:4). The final conjugated product was precipitated by the addition of cold ether, washed with ether, filtered, and dried under vacuum.

A known amount of conjugate was weighed and dissolved in dimethylsulfoxide (DMSO). The extent of conjugation was determined by measuring the absorbance of the solution at 480 nm (wavelength for doxorubicin absorbance). A standard curve of absorbance of a series of doxorubicin concentrations in DMSO was used to determine the doxorubicin amount in the conjugate. The yield of the conjugation reaction was ˜90%.

(B) Synthesis of Nanocores and Scanning Electron Microscopy of the Nanocores (FIG. 3B).

Nanocores were formulated using an emulsion-solvent evaporation technique. Briefly, 50 mg PLGA-DOX was allowed to dissolve completely in 2.5 mL acetone for one hour at room temperature. At this time, 0.5 mL methanol was added and the entire solution was emulsified into an aqueous solution of PVA (0.5 g/25 mL) by slow injection with constant homogenization using a tissue homogenizer followed by one minute of sonication (Misonix, Farmingdale, N.Y.). The emulsion was added to a dilute aqueous solution of PVA (0.2 g/100 mL) with rapid mixing for 3 hours at room temperature to evaporate any residual acetone or methanol. Nanocore size fractions were recovered by ultracentrifugation at 8,000, 15,000, 20,000, and 22,000 RPMs. Nanocores from the smallest size fractions were extruded through a 100 nm membrane using a hand-held extruder (Avestin, Ottawa, ONT) to obtain nanocores for encapsulation within nanocells. The nanocores were sized by dynamic light scattering (Brookhaven Instruments Corp, Holtsville, N.Y.) as well as by SEM (FIGS. 3B and 3E). For SEM preparation, nanocores were lyophilized for 72 hours following which a small quantity was dusted onto a carbon grid and coated with gold. Particles were analyzed using a Philips EM at a magnification of 65000×. All nanocores were used within 2 hours of synthesis to minimize aggregation.

To prepare the surrounding matrix/nanoshell, cholesterol (CHOL), egg-phosphatidylcholine (PC), and distearoylphosphatidylcholine-polyethylene glycol (m.w. 2000) (DSPE-PEG) were obtained from Avanti Polar Lipids (Birmingham, Ala.). Combretastatin A4 was obtained from Tocris Cookson (Ellisville, Mo.). All other reagents and solvents were of analytical grade.

PC:CHOL:DSPE-PEG (2:1:0.2 molar) lipid membranes were prepared by dissolving 27.5 mg lipid in 2 mL chloroform in a round bottom flask. 12.5 mg of combretastatin A4 was co-dissolved in the choloroform mixture at a 0.9:1 drug:lipid molar ratio. Chloroform was evaporated using a roto-evaporator to create a monolayer lipid/drug film. This film was resuspended in 1 mL H₂O after one hour of shaking at 65° C. to enable preferential encapsulation of combretastatin A4 within the lipid bilayer. The resulting suspension was extruded through a 200 nm membrane at 65° C. using a hand held extruder (Avestin, Ottawa, ONT) to create unilamellar lipid vesicles. The average vesicle size was determined by dynamic light scattering (Brookhaven Instruments Corp, Holtsville, N.Y.). Encapsulation efficiency was determined by passage of the drug/lipid mixture through a PD-10 column containing Sephadex G-25 (Pharmacia Biotech) with UV monitoring of combretastatin A4 elution at 290 nm.

PLGA-DOX nanocores were prepared as described above, and nanocores 100 nm were selected for encapsulation in nanocells by extrusion through a 100 nm membrane. When synthesizing CHOL:PC:DSPE-PEG:Combretastatin nanocells, nanocores containing 250 kg doxorubicin were added to the aqueous lipid resuspension buffer. The mixture was analyzed using TEM to determine encapsulation efficiency. The nanocores were lyophilized for 72 hours, following which a small quantity was dusted onto a carbon grid and coated with gold. They were analyzed using a Philips EM at a magnification of 65000× (FIG. 3B).

(C) Synthesis and transmission electron micrographs of nanocells (FIG. 3C). The sample was fixed in 2.5% gluteraldehyde, 3% paraformaldehyde with 5% sucrose in 0.1M sodium cacodylate buffer (pH 7.4), embeded in low temperature agarose and post fixed in 1% OsO4 in veronal-acetate buffer. The sample was stained in block overnight with 0.5% uranyl acetate in veronal-acetate buffer (pH6.0). Then dehydrated and embedded in epon-812 resin. Sections were cut on a Leica ultra cut UCT at a thickness of 70 nm using a diamond knife, stained with 2.0% uranyl acetate followed by 0.1% lead citrate and examined using a Philips EM410. Dynamic laser light scatter experiments also confirmed the size range to be between 180-220 nm (FIGS. 3D and 3E).

(D) Physicochemical release kinetics studies. Concentrated drug-loaded nanocells were suspended in 1 ml of PBS or hypoxic cell lysate buffer, and sealed in a dialysis bag (M.W. cutoff: 10,000, Spectrapor). The dialysis bag was incubated in 20 ml of PBS buffer at 37 C with gentle shaking 200 ul aliquots were taken from the incubation medium at predetermined time intervals and stored frozen for analysis. Released drug was quantified by reverse phase HPLC using a C18 column (4.5 mm×150 mm, Waters) with acetonitrile (A) and water (B) as eluents. Starting conditions were 80% A and 20% B with a linear gradient over 15 min to 10% A and 90% B, a linear gradient over five minutes to 0% A and 100% B, and a linear gradient over 5 min returning to the start conditions with a flow rate of 1 ml/min. A standardized amount of dexamethasone was added as an internal control for absolute quantification of combretastatain A4 and doxorubicin. Combretastatin A4 and dexamethasone were detected by wavelength monitoring at 295 nm and doxorubicin was detected by wavelength monitoring at 480 nm. Large quantities of combretastatin is released first from the nanocell followed by a prolonged and slow release of doxorubicin from the nanocore. The amount of free doxorubicin released is small as compared to the doxorubicin-PLGA fragments, emphasizing that free doxorubicin and the active doxorubicin-PLGA fragments, and not doxorubicin-PLGA oligomers, contribute to the cytotoxic effect (FIG. 3F).

### Example 2

### Developing the Novel In Vitro Assay System

Protocol: For setting up the system, human umbilical vein endothelial cells, pooled from three donors, were purchased from Cambrex, and used between passages 3-6. The cells were grown in endothelial basal medium supplemented with 20% fetal bovine serum (FBS) and bulletkit-2 (Sengupta et al. Cancer Res. 63(23):8351-59, Dec. 1, 2003). For the tumor component, we used B16/F10 melanoma cells as the model cell line, which were stably transfected to express green fluorescent protein. Plasmid expressing enhanced green fluorescent protein (pEGFP-C2, Clontech) was linearized and lipofected (Lipofectamine 2000, Invitrogen) into B16-F10 cells. The stably integrated clones of B16-F10 cells were selected by 800 μg/ml G418. The green fluorescence of the G418 resistant clones was further confirmed by Flow Cytometry and epifluorescence microscopy. The GFP-B16/F10 cells were regularly cultured in DMEM supplemented with 5% FBS. Sterile glass coverslips (Corning) were coated with matrigel (extracellular matrix extracted from murine Englebreth-Holms sarcoma, diluted 1:3 in phosphate buffer saline; Becton Dickinson) or collagen (type I from rat's tail, Becton Dickinson). Synchronized human umbilical vein endothelial cells were trypsinised and plated on the coverslips at a density of 2×10⁴ cells per well. The cells were allowed to adhere for 24 hours in endothelial basal media supplemented with 20% fetal bovine serum. At this time point, the media was replaced with EBM supplemented with 1% serum, and green fluorescent protein-expressing B16/F10 cells were added to the system at a density of 5×10³ cells per well. The co-culture was allowed to incubate overnight, following which different treatments were added to the media. At 24 hours post-treatment, the cells were fixed in paraformaldehyde (4% on ice, for 20 min), and stained with propidium iodide. The coverslips were mounted with antifade, and analysed with a LSM510 Zeiss confocal microscope. The fluorochromes were excited using 488 nm and 543 nm laser lines, and the emitted light was captured using 505/30 nm and 565/615 band pass filters. The images were captured at a resolution of 512×512 pixels. Quantification of the area covered by the endothelial cells or GFP-BL6/F10 cells was carried out using a planimetric point-count method using a 224-intersection point square reticulum. Data were expressed as the ratio of each component to the total area covered by cells.

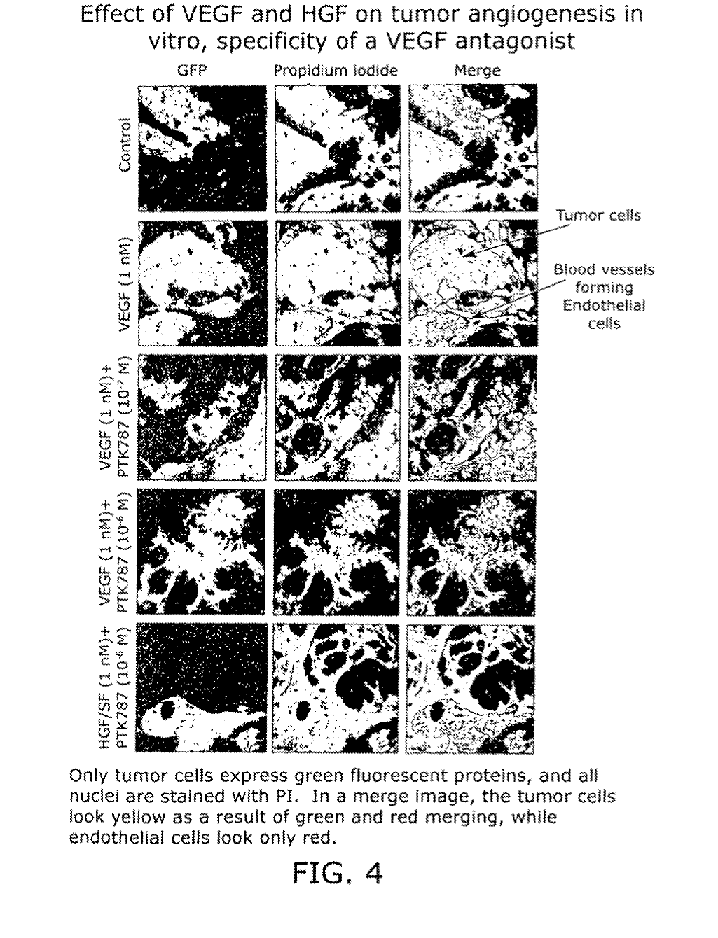

### Effect of VEGF and HGF on Tumor Angiogenesis In Vitro (FIG. 4)

Endothelial cells formed a limited number of tubular networks within 24 hours of plating on matrigel (1:3 dilution). However, the addition of tumor cells to establish the co-culture accelerated the tubulogenic process. The GFP+ tumor cells were visualized to concentrate into clusters surrounded and integrating with the vascular network. The addition of both VEGF and HGF/SF resulted in a significant increase in the vascular network. To validate the sensitivity of the system to elucidate the modulation of specific pathways, we used a VEGF receptor antagonist, PTK787. As expected, VEGF-induced angiogenesis was blocked by PTK787 at a concentration that had no effect on the HGF/SF-induced response (FIG. 4).

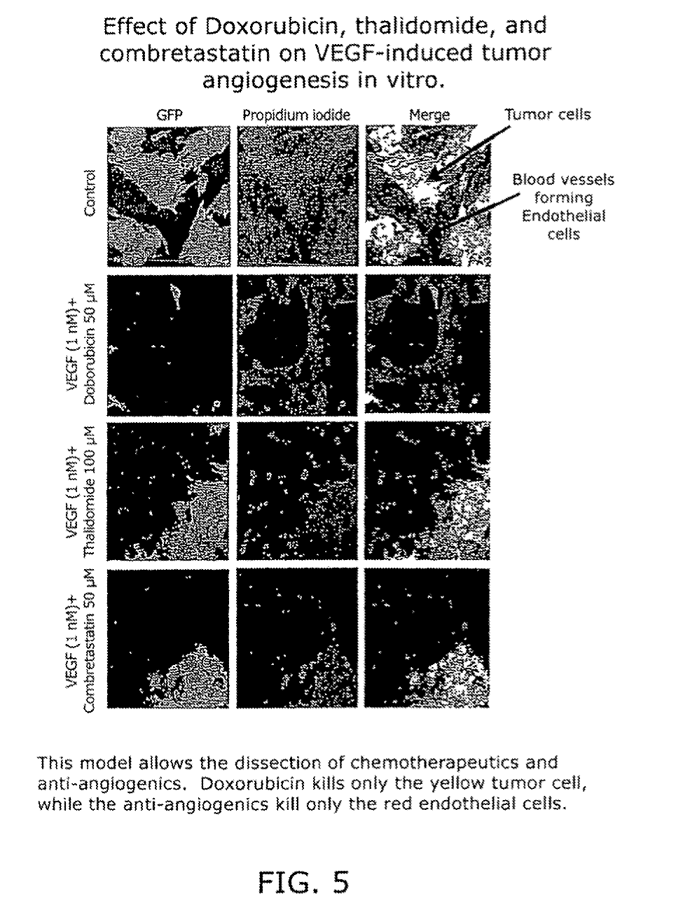

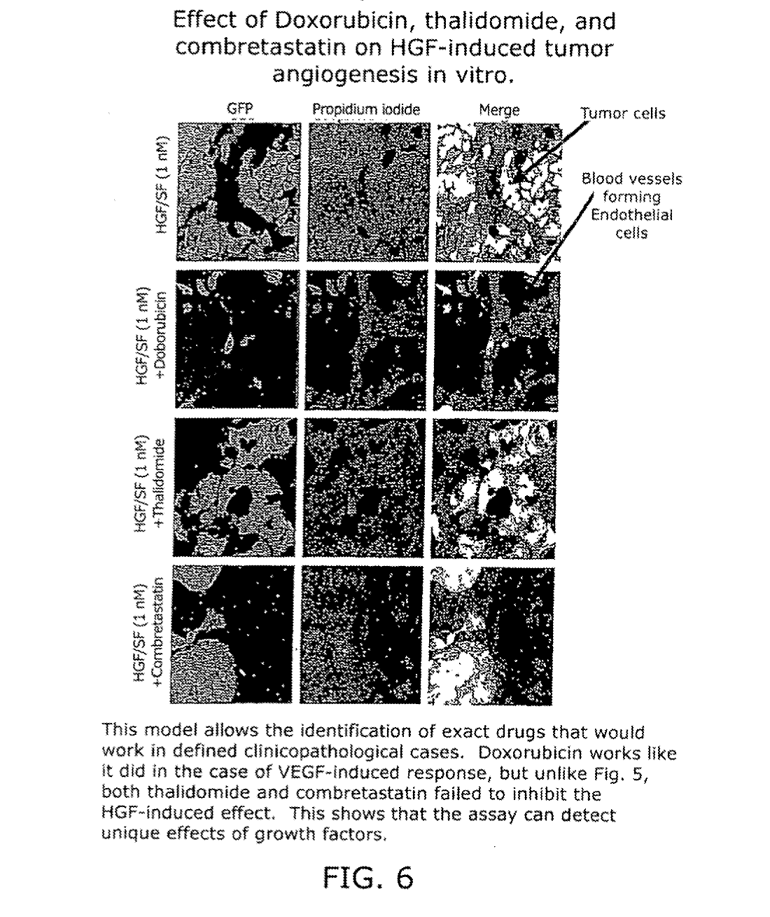

### Effect of Combretastatin, Thalidomide, and Doxorubicin on VEGF- or HGF/SF-Induced Response (FIGS. 5 & 6)

As shown in FIG. 5, incubation with Doxorubicin (10-50 μM) exerted a selective ablation of the tumor cells in a concentration-dependent manner. Even at the highest concentration used (50 mM), no effect on the VEGF-induced endothelial network was evident. In contrast, both thalidomide and combretastatin exerted a collapse of the VEGF-induced vascular network without affecting the tumor cells.

Similar to the VEGF-induced co-culture experiments, doxorubicin exerted a selective induction of tumor cell death in the presence of HGF/SF (FIG. 6). However, in contrast to VEGF, HGF/SF prevented the ablation of endothelial cellular network in the presence of thalidomide or combretastatin (FIG. 6). The susceptibility of VEGF-induced angiogenesis and the protective effect of HGF/SF against these two indirect anti-angiogenics indicate the functional difference at the level of intracellular signaling induced by the two growth factors.

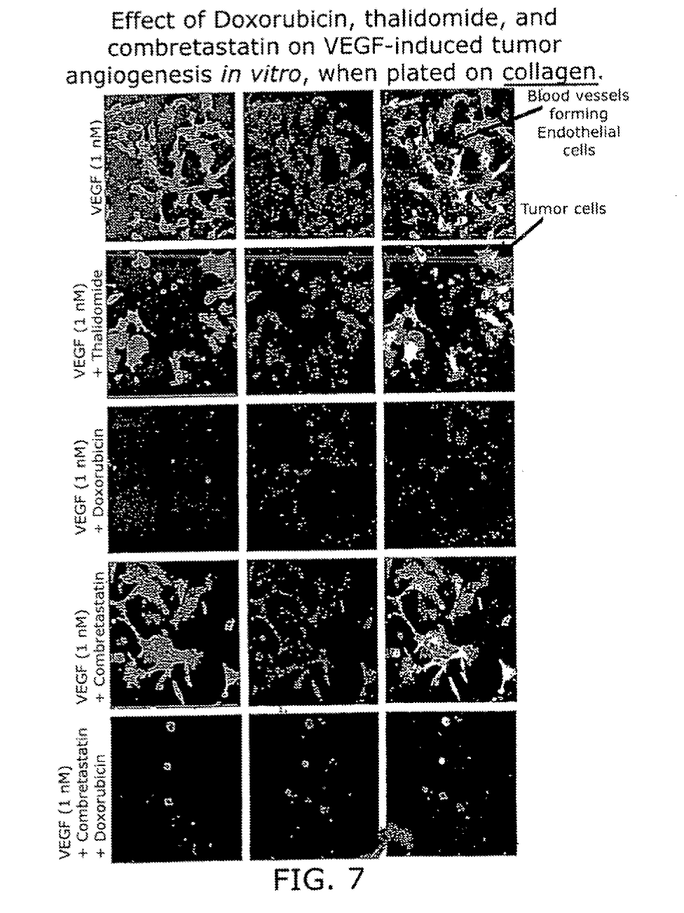

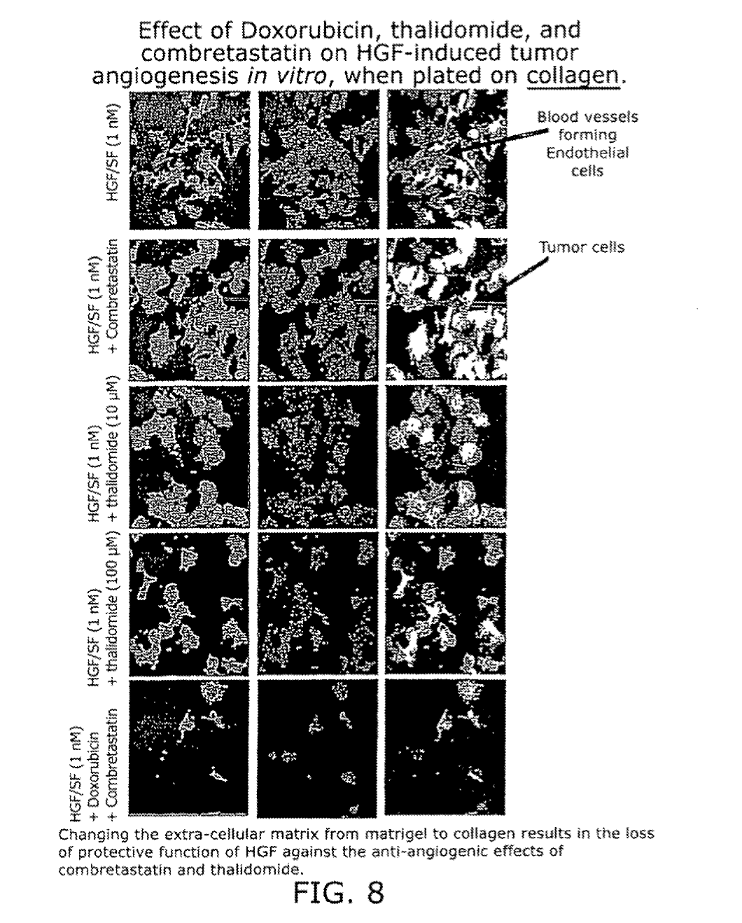

### Effect of Collagen Matrix on VEGF- or HGF-Induced Tumor Response (FIGS. 7-8)

Endothelial cells plated on collagen matrix assumed a flat ‘cobble-stone’ morphology unlike the tubular networks formed when plated on matrigel. Furthermore, the melanoma cells also assumed a ‘spreading-out’ morphology with the formations of focal adhesions, and did not form cell clusters as seen on matrigel. Incubation with doxorubicin induced tumor cell death in both VEGF- and HGF/SF-treated co-cultures (FIGS. 7, 8). As shown in FIG. 7, both combretastatin and thalidomide inhibited the angiogenic effects of VEGF. Intriguingly, the protective effect of HGF/SF that was observed on cells plated on matrigel was lost when the cells were plated on collagen, and both thalidomide and combretastatin induced endothelial cell loss (FIG. 8). The current findings emphasize the need to incorporate the extracellular component while screening for anti-angiogenic therapies.

### Example 3

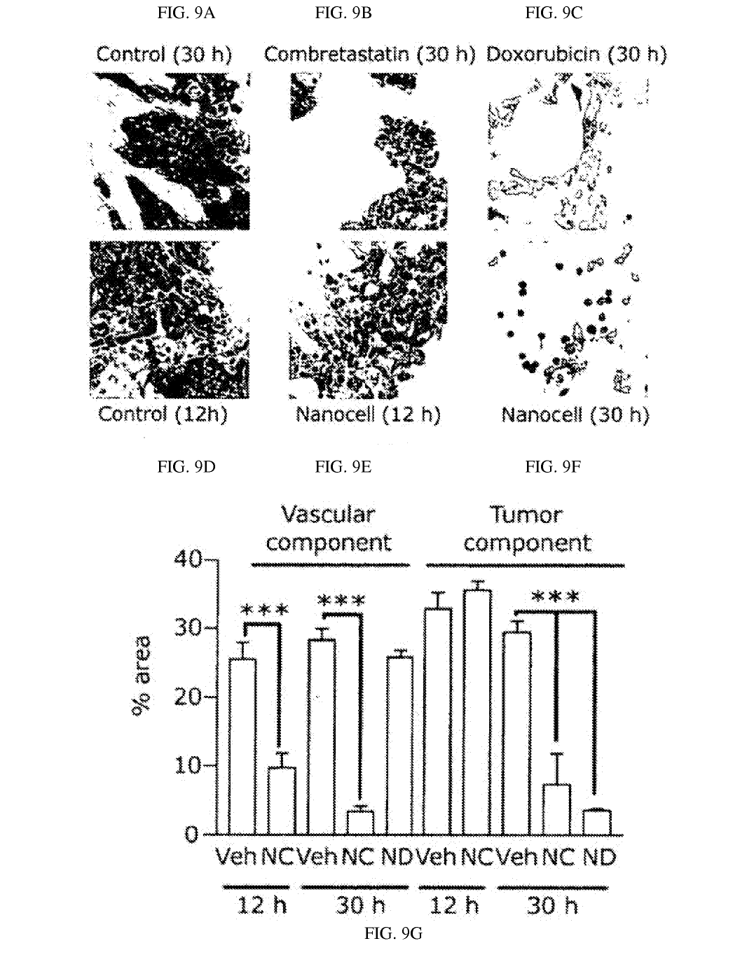

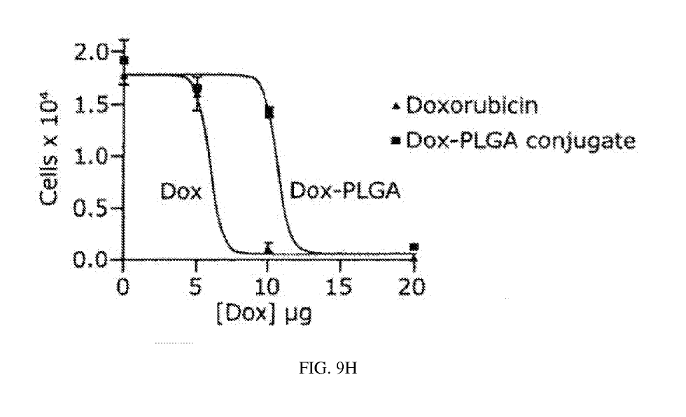

### In Vitro Efficacy of Drug Loaded Nanocells (FIG. 9)

Sterile glass coverslips (Corning) were coated with matrigel (extracellular matrix extracted from murine Englebreth-Holms sarcoma, diluted 1:3 in phosphate buffer saline; Becton Dickinson) or collagen (type I from rat's tail, Becton Dickinson). Synchronized human umbilical vein endothelial cells were trypsinised and plated on the coverslips at a density of 2×10⁴ cells per well. The cells were allowed to adhere for 24 hours in endothelial basal media supplemented with 20% fetal bovine serum. At this time point, the media was replaced with EBM supplemented with 1% serum, and green fluorescent protein-expressing B16/F10 cells were added to the system at a density of 5×10³ cells per well. The co-culture was allowed to incubate overnight, following which different treatments were added to the media. At 24 hours post-treatment, the cells were fixed in paraformaldehyde (4% on ice, for 20 min), and stained with propidium iodide. The coverslips were mounted with antifade, and analysed with a LSM510 Zeiss confocal microscope. The fluorochromes were excited using 488 nm and 543 nm laser lines, and the emitted light was captured using 505/30 nm and 565/615 band pass filters. The images were captured at a resolution of 512×512 pixels. Quantification of the area covered by the endothelial cells or GFP-BL6/F10 cells was carried out using a planimetric point-count method using a 224-intersection point square reticulum. Data were expressed as the ratio of each component to the total area covered by cells.

As shown in the pictographs, incubation with doxorubicin-loaded nanocores resulted in the selective loss of yellow-melanoma cells without affecting the angiogenic outcome. In contrast, the incubation with combretastatin entrapped in the surrounding lipid matrix resulted in a selective loss of the vascular network, demonstrating its selectivity against endothelial cells. When the co-culture was incubated with combretastatin and doxorubicin-loaded nanocells, it resulted in a rapid death of endothelial cells first followed by the complete loss of the entire co-culture. This demonstrated that in a simulation that closely mimics the pathophysiology, the active agent (Combretastatin in this case) in the surrounding matrix is released prior to the active agent linked to the nanocore (Doxorubicin for this example), emphasizing the spatio-temporal effect resulting from the use of the nanocell, and better efficacy since it results in complete ablation of the tumor.

### Example 4

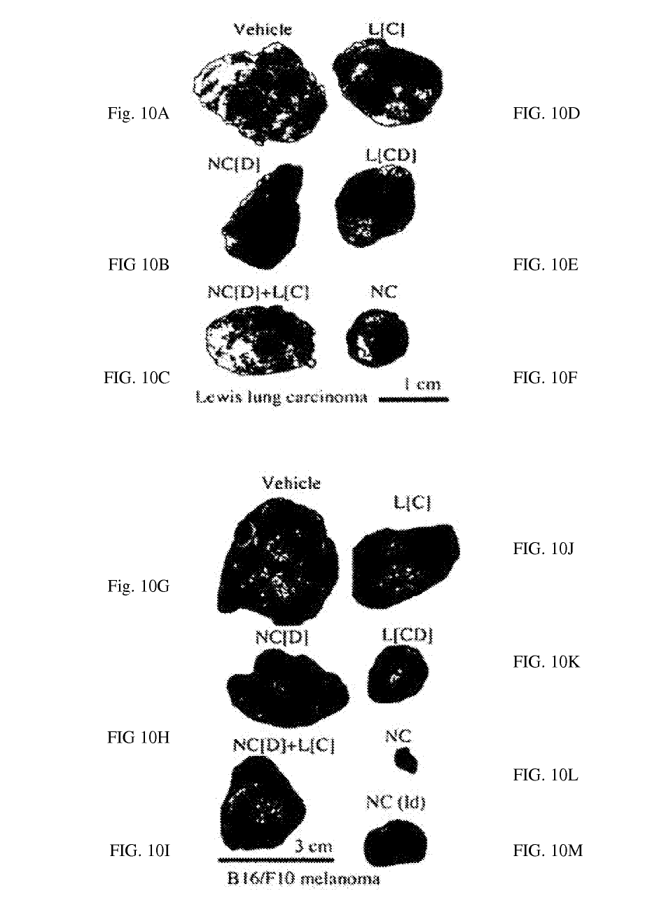

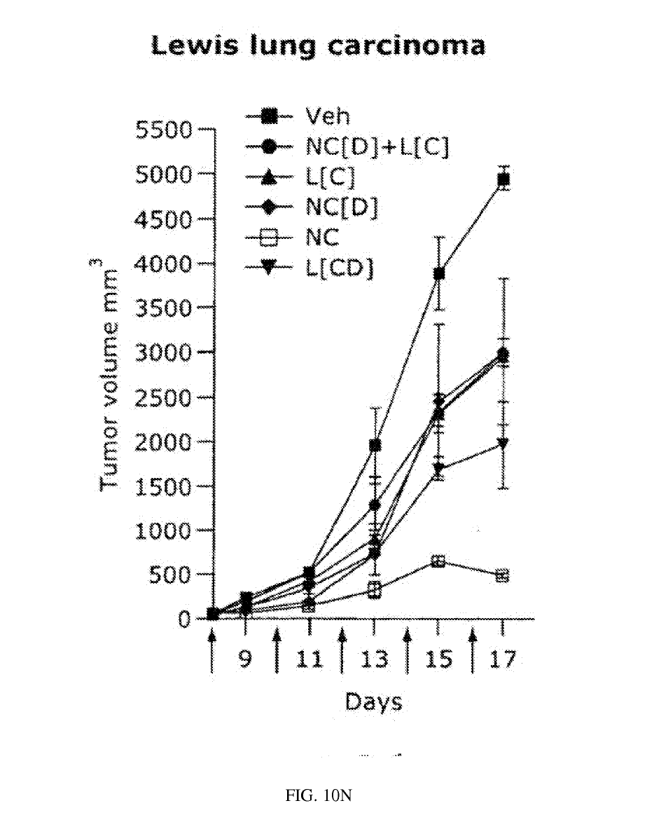

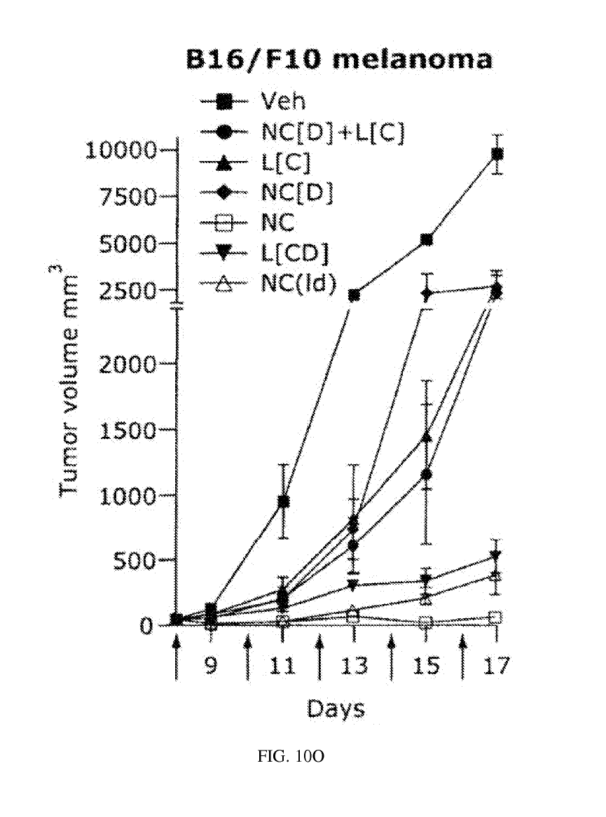

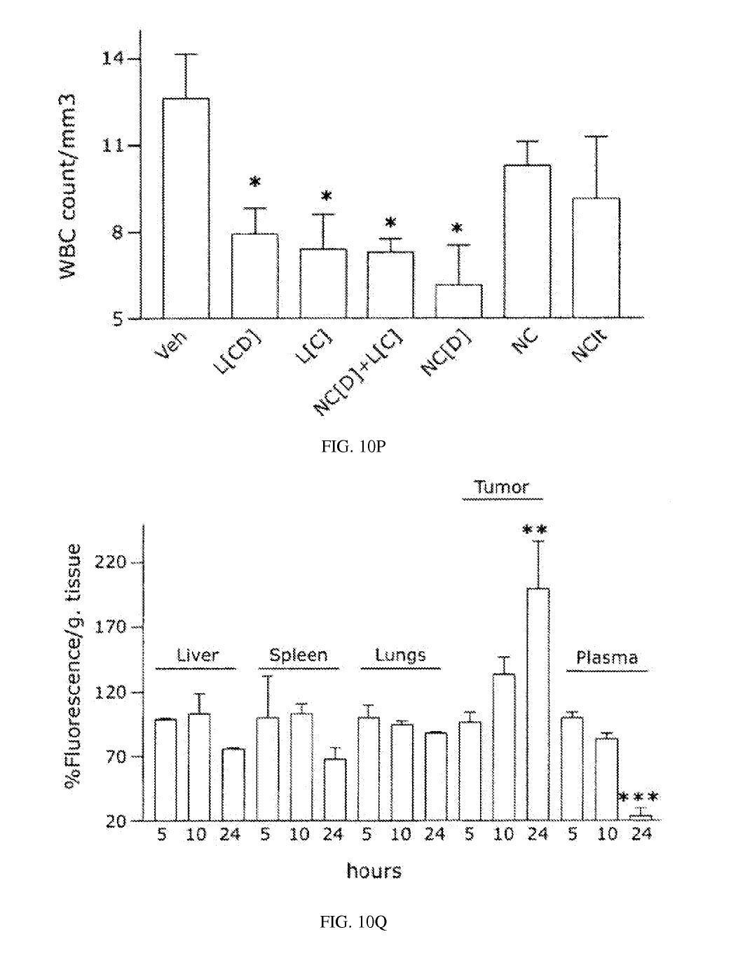

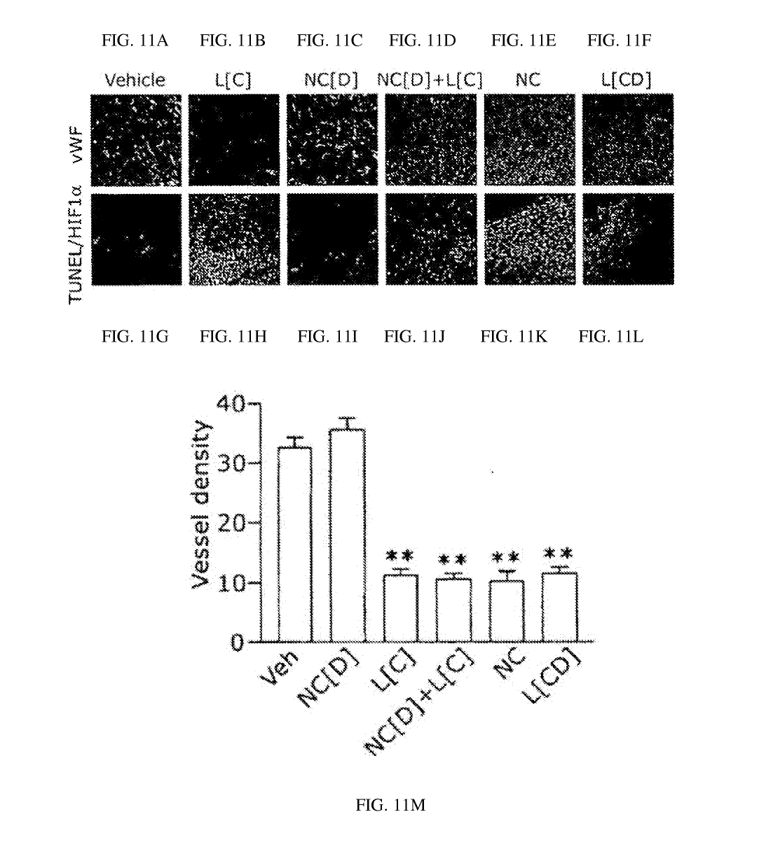

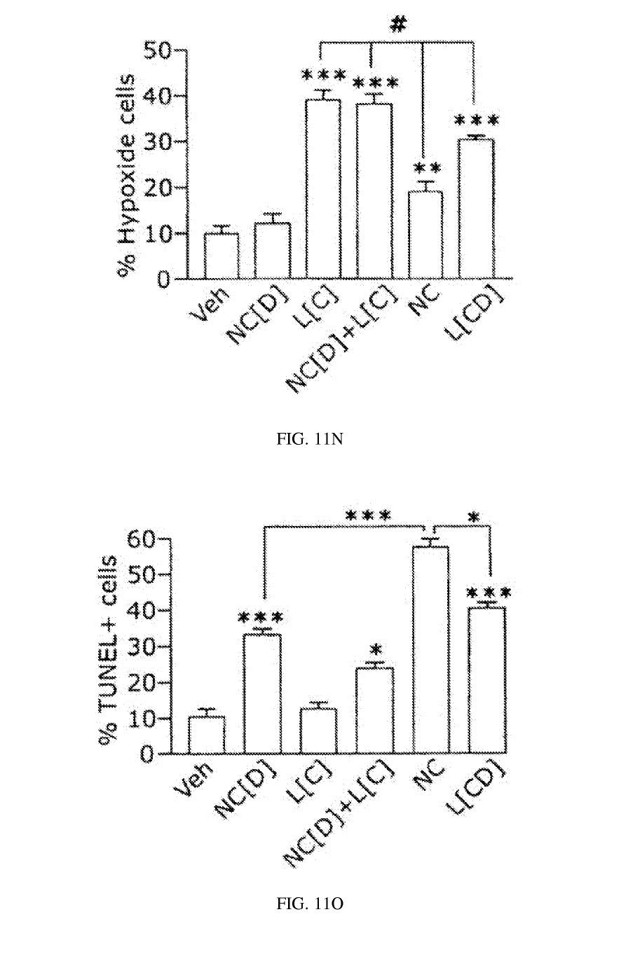

### In Vivo Tumor Model (FIG. 10)

Male C57/BL6 mice (20 g) were injected with 3×10⁵ YFP-BL6/F10 cells or 2.5×10⁵ Lewis Lung carcinoma cells into the flanks. The growth of the tumors was monitored regularly. The mice were randomized into different treatment groups when the tumor reached either 50 or 150 mm³ in volume. Treatment was administered through the tail vein, every alternate day, for 3-7 applications. The tumor dimensions were measured everyday, and the tumor volume was calculated according to the formula:

Volume=3.14/6×Length×Width².

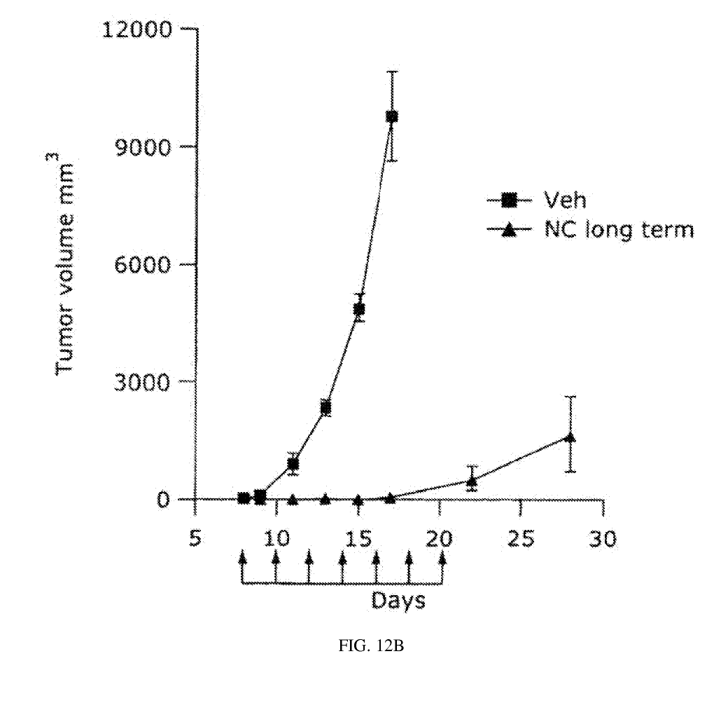

The animals were sacrificed at specific time points (see FIGS. 10 and 12), and the tumors were photographed for gross morphology, and excised for histopathological analysis. Simultaneously, 1 ml of blood was drawn through cardiac puncture, and analyzed for toxicity profile of the treatment regimens, since white blood cell counts are most susceptible to the effects of chemotherapeutics.

The photographs demonstrate the effect of different formulations of drugs and combinations on melanoma growth in mice as compared with the nanocell-treated group. Treatment with both doxorubicin-nanocores and nano lipid-entrapped Combretastatin resulted in the reduction of tumor proliferation, with an additive effect when combined together. However, when administered in the nanocell formulation, the outcome was significantly superior to any of the comparative groups. This supports our hypothesis that the nanocell delivers the dox-nanocore into the tumor prior to the disruption of the vasculature.

The graphs show the effect of different treatments of the differential blood count and hemoglobin levels. The least toxicity was observed with the Nanocell-treated group, despite the fact that it was most potent, suggesting that the chemotherapeutic agent (Doxorubicin) is trapped within the tumor and less quantity can leak out into the systemic circulation as the vessels are collapsed prior to its release from the nanocore.

### Example 5

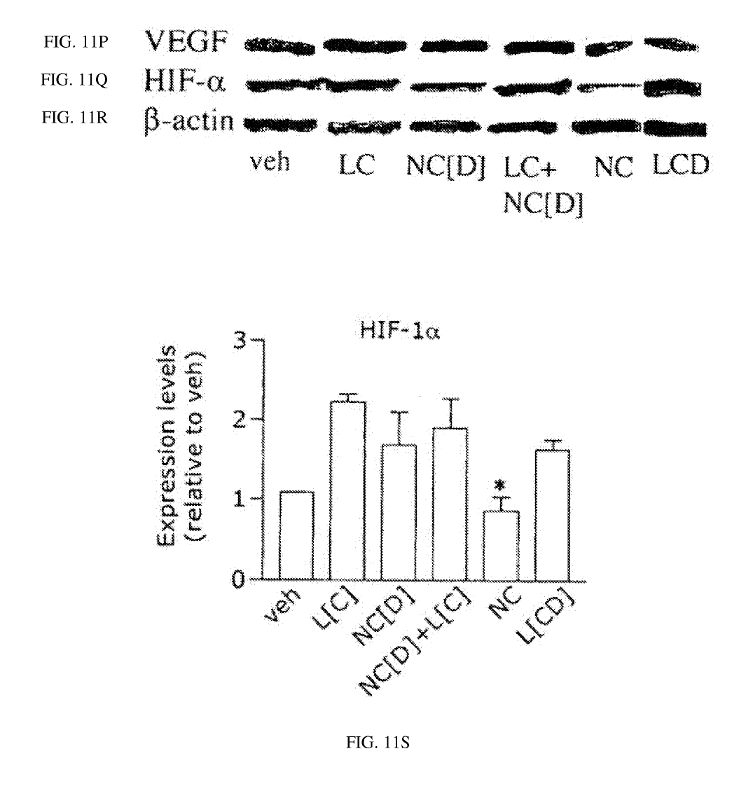

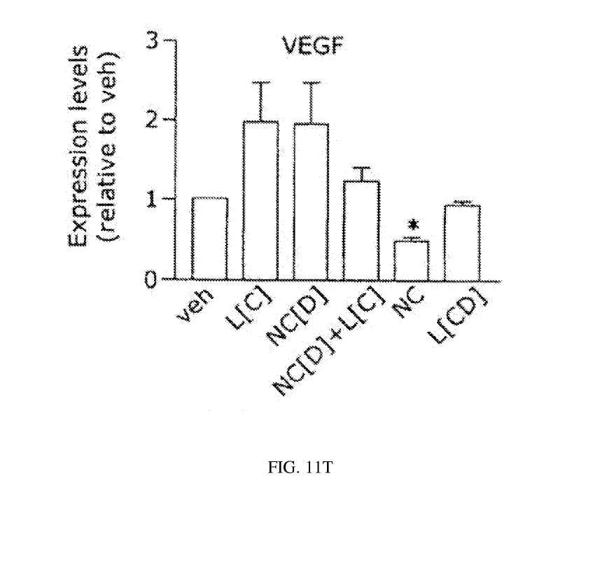

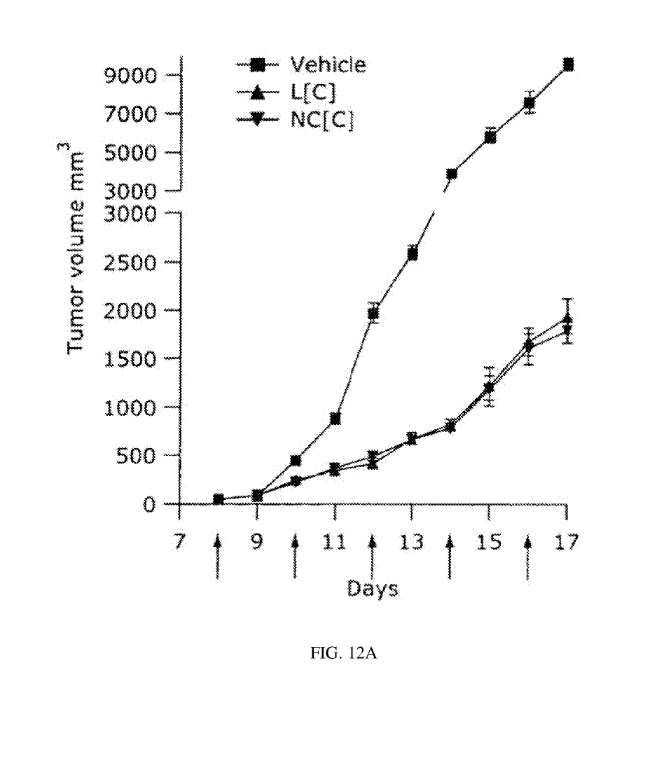

### Effect of Different Treatment on the Tumor Neovasculature (FIG. 11)

The treatment with Nanocore-Doxorubicin (ND) has no effect on the vasculature or the vessel density (see graph), while nano lipid-micellar Combretastatin (LC) reduces the vessel density as well as collapses the vasculature. Although, ND+LC was synergistic, no significant difference existed between this group and that achieved using the nanocell. This is expected since in both groups, LC is expected to work earlier than ND.

### Example 6

### Effect of Different Treatment on the Tumor Apoptosis. (FIG. 12)

Cells undergoing apoptosis are stained red as they are TUNEL positive. Although, LC+ND and the nanocell-treated groups had the same effect on the tumor vasculature, it is evident that the latter induced greater apoptosis in the tumor. This explains the better therapeutic outcome observed in the nanocell-treated group, and also supports the hypothesis that the Doxorubicin is released from the nanocores, which are trapped within the tumor as a result of the LC-mediated collapse of the tumor vessels. In contrast, LC+ND-treated sections show lesser apoptosis since the vessels are collapsed prior to the entry of significant quantity of ND into the tumor stroma.

### Example 7

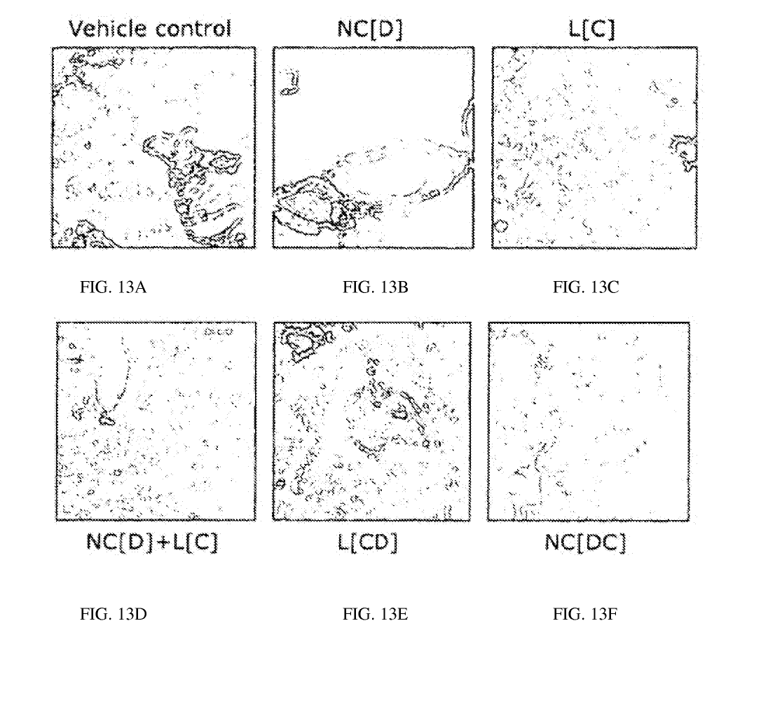

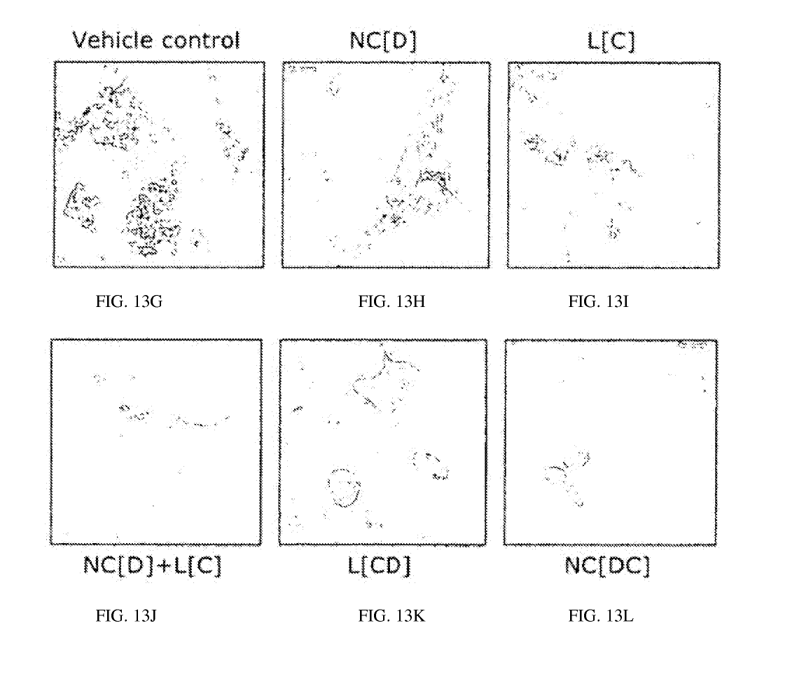

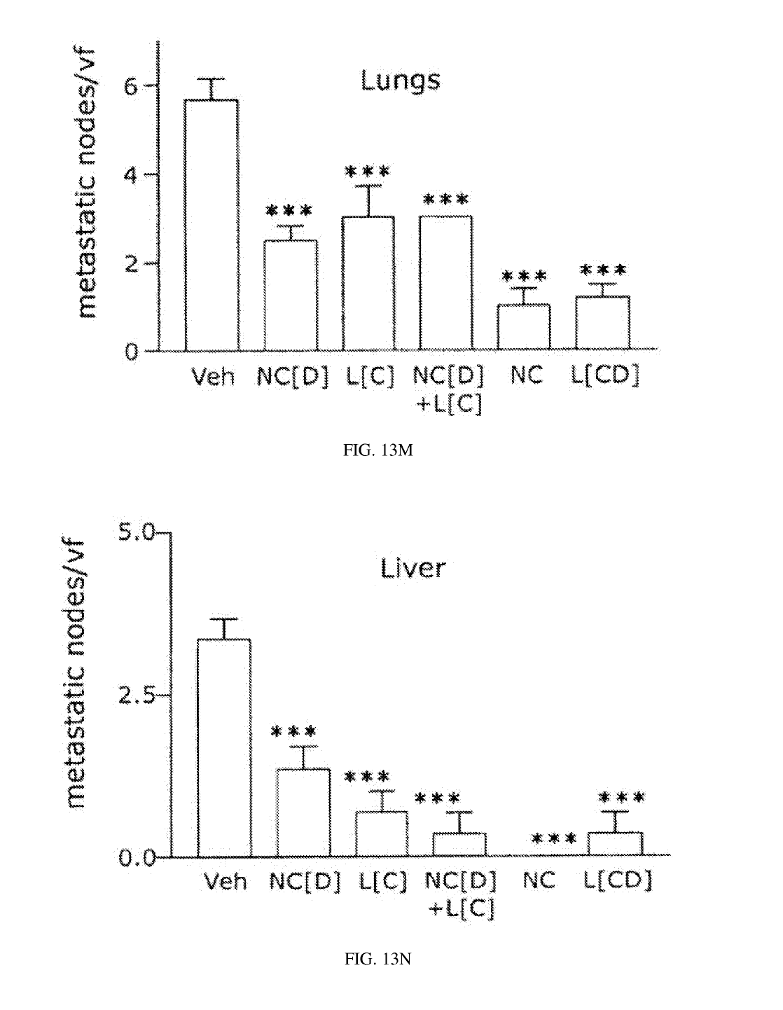

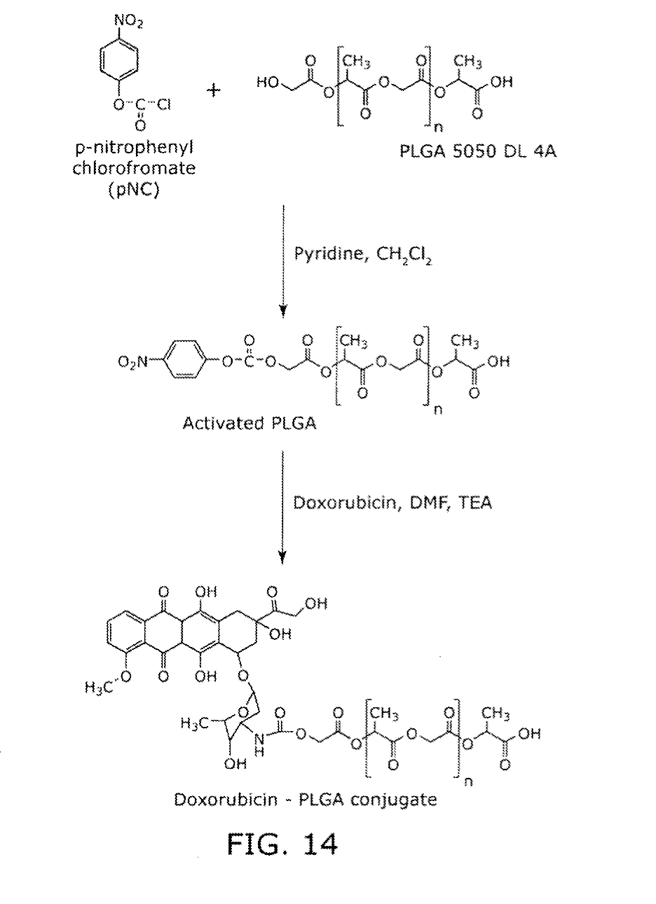

### Effect of Different Treatments on Metastasis (FIG. 13)

Melanoma is an aggressive tumor that spontaneously metastasizes to the liver and the lungs besides other organs. We evaluated the effect of different treatment conditions on the metastasis to lungs (upper panel set) and liver (Lower panel set), by evaluating the number of metastatic nodes in these organs. This was done by counting the number of green fluorescent-positive nodes, although in the pictographs they appear as yellow from the merging of green fluorescent with the red emission from all cells that were labeled with a dye that labels the nuclei. As shown, the treatment with nanocell prevented metastasis to both the organs.

### Example 8

### Tissue Distribution Studies

Nanocells were synthesized loaded with fluorescein dye. Free fluorescein was removed by passing the nanocells through a Sephadex G25 column. The fluorescein-nanocells were injected into tumor-bearing mice. The animals were sacrificed at 5, 10, and 24 hours post-administration. Serum, tumor, liver, lungs, and spleen were collected during necropsy, and fluorescein was extracted from these tissues using methanol. The amount of fluorescein in each sample was detected using a fluorescence plate reader, and normalized to the tissue weight. The nanocells clearly accumulated in the tumor and not in other organ systems (FIG. 10F).

### Example 9

### Nanocells for Treatment of Asthma

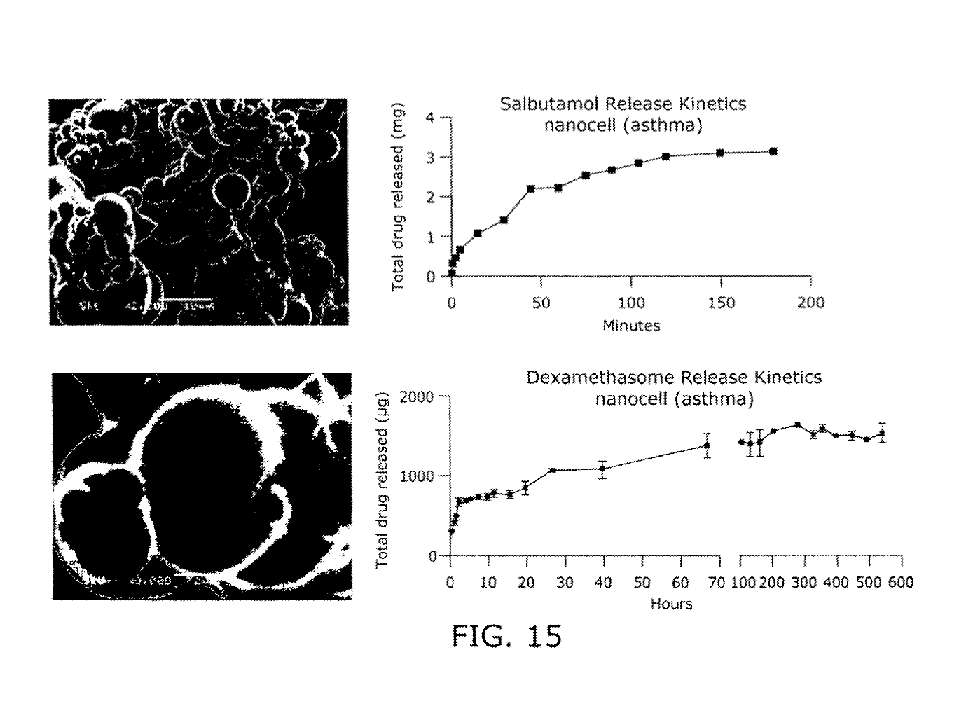

FIG. 15 shows the structure and release kinetic profile of nanocells developed for treatment of asthma. The electron micrograph shows the ultrastructure of nanocells where the biodegradable-nanocore is coated with a lactose shell. A corticosteroid (anti-inflammatory agent) is entrapped within the nanocore, while a bronchodilator is entrapped in the lactose matrix surrounding the nanocore. The graphs demonstrate the fact that the bronchodilator (salbutamol) is released first in a time scale of minutes, while the corticosteroid (dexamethasone) is released in a slow prolonged manner. This temporal release would enable the constricted bronchioles during asthma to get dilated first allowing the permeation of the nanocores into deeper lung. The subsequent slow release would block the chronic inflammation that follows an acute asthma episode.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

We claim:

1. A nanoparticle comprising a polyester covalently linked to an anti-neoplastic agent, wherein the polyester has a molecular weight of 100-20,000 g/mol, and wherein said nanoparticle 10-1000 nm in its greatest diameter.

2. The nanoparticle of claim 1, wherein the nanoparticle is 20-800 nm in its greatest diameter.

3. The nanoparticle of claim 1, wherein the nanoparticle is 50-500 nm in its greatest diameter.

4. The nanoparticle of claim 1, wherein said polyester is coupled to said anti-neoplastic agent by a reaction in which a nucleophile attacks an electrophile.

5. The nanoparticle of claim 1, wherein said polyester is coupled to said anti-neoplastic agent by a reaction in which a hydroxyl group attacks an activated carbonyl.

6. The nanoparticle of claim 1, wherein the polyester comprises monomers of glycolic acid.

7. The nanoparticle of claim 1, further comprising a copolymer.

8. The nanoparticle of claim 7, wherein the copolymer comprises a hydrophobic block and hydrophilic block.

9. The nanoparticle of claim 8, wherein the hydrophobic block is a polyester.

10. The nanoparticle of claim 8, wherein the hydrophilic block is polyethylene glycol.

11. The nanoparticle of claim 1, further comprising a lipid.

12. The nanoparticle of claim 11, wherein the lipid is cholesterol.

13. The nanoparticle of claim 11, wherein the lipid is phosphatidylcholine.

14. The nanoparticle of claim 11, wherein the lipid is 1 palmitoyl phosphatidtylcholine.

15. The nanoparticle of claim 1, wherein said anti-neoplastic agent is doxorubicin.

16. The nanoparticle of claim 1, wherein said anti-neoplastic agent is tamoxifen.

17. The nanoparticle of claim 1, wherein said anti-neoplastic agent is taxol.

18. A drug delivery particle for the delivery of two different therapeutic agents, comprising
a nanocore including a first therapeutic agent; and
an outer layer coating said nanocore, said outer layer including a second therapeutic agent, wherein the second therapeutic agent is released first, followed by the release of the first therapeutic agent from the nanocore.

19. A drug delivery particle for temporal delivery in a patient of two different therapeutic agents, comprising
a nanoparticle comprising a polymeric matrix containing a first therapeutic agent, which first therapeutic agent is released upon the dissolution or degradation of said polymeric matrix; and
an outer layer coating said nanoparticle, said outer layer including a second therapeutic agent and which outer layer allows a fast release of said second therapeutic agent, wherein the second therapeutic agent is released first, followed by a slower release of the first therapeutic agent from the nanoparticle, such that the effect of the second therapeutic agent begins before the first therapeutic agent reaches therapeutic levels in the patient.
